(19) United States
(12) Patent Application Publication
(10) Pub. No.: US 2026/0224535 A1
(43) Pub. Date: Aug. 6, 2026

(54) TREATMENT FOR AUDITORY DISORDERS

(71) Applicants: Douglas Bennion, Iowa City, IA (US); Marlan Hansen, Iowa City, IA (US); Zachary Urdang, Iowa City, IA (US); Carolina Chu, Iowa City, IA (US)

(72) Inventors: Douglas Bennion, Iowa City, IA (US); Marlan Hansen, Iowa City, IA (US); Zachary Urdang, Iowa City, IA (US); Carolina Chu, Iowa City, IA (US)

(21) Appl. No.: 19/468,862

(22) Filed: Feb. 3, 2026

Related U.S. Application Data
(60) Provisional application No. 63/753,057, filed on Feb. 3, 2025.

(51) Int. Cl. A61K 31/4178; A61K 31/437; A61K 38/08; A61K 38/10; A61K 38/16; A61P 27/16

(52) U.S. Cl. CPC A61K 31/4178; A61K 31/437; A61K 38/08; A61K 38/085; A61K 38/10; A61K 38/16; A61P 27/16

(57) ABSTRACT

Provided herein are medical treatments for auditory disorders, specifically methods utilizing angiotensin receptor blockers (ARBs), calcium channel blockers (CCBs), angiotensin II type 2 receptor (AT2R) agonists, and Mas receptor (MasR) agonists to prevent and treat conditions such as hearing loss, tinnitus, and Meniere's Disease. The disclosed methods address the lack of FDA-approved therapies for these disorders by repurposing safe, cost-effective medications traditionally used for hypertension and other conditions.

### PRIORITY

This application claims the benefit of the filing date of U.S. provisional application Ser. No. 63/753,057, filed on Feb. 3, 2025, the disclosure of which is incorporated by reference herein.

### GOVERNMENT SUPPORT

This invention was made with government support under DC018488 and DC000242 awarded by the National Institutes of Health. The government has certain rights in the invention.

### FIELD OF INVENTION

The present disclosure pertains to medical treatments for auditory disorders, specifically methods utilizing angiotensin receptor blockers, calcium channel blockers, angiotensin II type 2 receptor agonists, and Mas receptor agonists for the prevention and treatment of conditions such as hearing loss, tinnitus, and Meniere's Disease.

### BACKGROUND

One in five people worldwide suffers from auditory disorders. There are currently no FDA approved therapies for three of the major diseases that make up the majority of auditory disorders: hearing loss, tinnitus, and endolymphatic hydrops (e.g. Meniere's Disease). Despite off-label use of other treatments, these conditions are largely treated with supportive management and ultimately with surgery when indicated (e.g., cochlear implantation, endolymphatic sac decompression/shunting).

### SUMMARY

Provided herein are novel uses of medications for the treatment of auditory disorders, for example, those that target the renin angiotensin system (RAS; which medications of which have been time-tested and are proven to be safe over decades of use in millions upon millions of patients for other indications (e.g., hypertension, chronic kidney disease, heart failure)). They also represent a promising treatment for sudden sensorineural hearing loss, the cause of which remains unknown, but that would benefit from the anti-inflammatory and vascular protective effects of RAS and other drugs. They are also low cost, and most RAS drugs having been off patent for many years. Repurposing of these medications for the treatment of auditory disorders represents an important and novel use that provide immediate impact in clinical care.

Additionally, provided herein is the use of AT2R agonists and Mas receptor agonists for the treatment of auditory disorders. AT2R agonists and Mas receptor agonists are currently in development for other unrelated indications (e.g., AT2R agonist buloxibutid is currently in Stage III trials for the treatment of pulmonary fibrosis).

In short, there are currently no approved medical treatments for auditory disorders, and existing off label treatments often leave patients with poorly controlled and significant symptoms, loss of function, and disability. Introduction of novel pharmacologic treatments for this burgeoning patient population is a great advantage in a field where no medications are routinely able to be employed.

Aspects of the invention include methods to prevent or treat an auditory disorder comprising administering to a subject in need thereof an angiotensin receptor blocker (ARB), a calcium channel blocker (CCB), an angiotensin II type 2 receptor agonist (AT2R), an angiotensin converting enzyme (ACE) inhibitor, a Mas receptor (MasR) agonist or a combination thereof. In some embodiment the ARB is not telmisartan. Another aspect includes methods to prevent or treat tinnitus, Meniere's Disease or dizziness/vertigo comprising administering to a subject in need thereof an angiotensin receptor blocker (ARB), a calcium channel blocker (CCB), an angiotensin converting enzyme (ACE) inhibitor, an angiotensin II type 2 receptor agonist (AT2R), a Mas receptor (MasR) agonist or a combination thereof. Aspects of the invention include methods to prevent or treat an auditory disorder comprising administering to a subject in need thereof an angiotensin II type 2 receptor agonist (AT2R). In some aspects, the auditory disorder is hearing loss, tinnitus, synaptopathy, Meniere's Disease, endolymphatic hydrops or an auditory processing disorder. In some aspects, the hearing loss is due to acoustic/noise trauma or ototoxicity. In some aspects, the hearing loss is sensorineural hearing loss. In some aspects, the angiotensin receptor is angiotensin II type 1 receptor (AT1R), angiotensin II type 2 receptor (AT2R) agonist, Mas receptor (MasR) agonist or combination thereof. In some aspects, the ARB is Azilsartan, Losartan, Valsartan, Candesartan, Telmisartan, Irbesartan, Olmesartan, Fimasartan, Saprisartan, Eprosartan or a combination thereof. In some aspects, the ARB is losartan. In some aspects, the AT2R agonist is buloxibutid, Compound 21, M024, CGP42112, PD-123-319, β-Pro7Ang III, angiotensin-(1-7), alamandine, and [Sar1,Ile8]Angiotensin II, or a combination thereof. In some aspects, CCB is Amlodipine, Aranidipine, Azelnidipine, Barnidipine, Benidipine, Cilnidipine, Clevidipine, Efonidipine, Felodipine, Isradipine, Lacidipine, Lercanidipine, Manidipine, Nicardipine, Nifedipine, Nilvadipine, Nimodipine, Nisoldipine, Nitrendipine, Pranidipine, Fendiline, Gallopamil, Verapamil, Diltiazem, Mibefradil, Bepridil, Flunarizine, Fluspirilene, Gabapentin, Pregabalin, Ziconotide or combination thereof. In some aspects, the Mas receptor (MasR) agonist is Angiotensin-(1-7), [D-Ala7]-Angiotensin-(1-7), p-Amino-phenylalanine-containing analogues of Angiotensin-(1-7), HPβCD-Angiotensin-(1-7), cyclodextran-bound angiotensin-(1-7), BIO101, CGEN-856S, CGEN-857, AVE0779, and CGP42112 or combinations thereof.

Some aspects provide methods to prevent or treat a shift in auditory brainstem response (ABR) from noise induced trauma comprising administering to a subject in need thereof an angiotensin receptor blocker (ARB), an angiotensin II type 2 receptor (AT2R) agonist, a calcium channel blocker (CCB), an angiotensin converting enzyme inhibitors (ACEi), a Mas receptor agonist or a combination thereof. Some aspects provide methods to prevent or treat a shift in auditory brainstem response (ABR) from noise induced trauma comprising administering to a subject in need thereof an angiotensin II type 2 receptor (AT2R) agonist.

In some aspects, the subject has hypertension. In other aspects, the subject does not have hypertension. In some aspects, the administration is oral or in film or coating on an implantable device. In some aspects, the administration is carried out hours, days, weeks, months, years prior to or after the onset of an auditory disorder.

### BRIEF DESCRIPTION OF THE DRAWINGS

The drawings illustrate generally, by way of example, but not by way of limitation, various embodiments discussed herein.

FIGS. 1A-1C. Losartan-infused chow results in measurable serum levels of (A) losartan and its metabolite (B) losartan-COOH at 1 day after noise-induced hearing loss as assessed by liquid chromatography-tandem mass spectrometry. (C) Phenacetin was added to the serum of mice from both groups as a positive control. Ctl=control chow group, n=12. Los=losartan chow group, n=11.

FIGS. 2A-2H. Pre-treatment with losartan protects against temporary shifts in auditory brainstem response (ABR) thresholds after mild noise-induced trauma without significantly affecting distortion product otoacoustic emissions (DPOAEs). ABR thresholds among mice treated with control versus losartan-infused chow (n=7/group) at baseline (A), and 1, 7, and 14 days (B-D) after noise exposure. DPOAE thresholds at baseline (E), and 1, 7, and 14 days (F-H) after noise exposure.

FIGS. 3A-3H. Pre-treatment with losartan protects against permanent shifts in auditory brainstem response (ABR) thresholds after moderate noise-induced trauma but does not significantly affect distortion product otoacoustic emissions (DPOAEs). ABR thresholds at baseline (A), and 1, 7, and 14 days (B-D) after noise exposure. DPOAE thresholds at baseline (E), and 1, 7, and 14 days (F-H) after noise exposure.

FIGS. 4A-4D. Losartan pretreatment preserves auditory brainstem response (ABR) Wave 1 amplitude growth and latencies after moderate noise-induced trauma. Mouse ABR wave 1 amplitude growth function at baseline (A) and at 14 days after noise exposure (B) and wave 1 latencies at the same points (C, D).

FIGS. 5A-5G. Losartan pretreatment protects against cochlear synaptopathy after mild noise-induced trauma. (A&B) Representative whole mount immunostaining of cochlear hair cells and synapses in (A) control and (B) losartan-treated groups. (C&D) Representative high power confocal images of inner hair cell (IHC) synapses at the basal turn of the cochlea from (C) control and (D) losartan-treated mice. (E) Average number of synapses per IHC in control and losartan groups after mild noise exposure causing a temporary threshold shift. (F&G) Number of IHCs or OHCs per 100 μm of arc-length in control and losartan treatment groups in the basal, middle, and apical cochlear turns. CtBP2 (pre-synaptic ribbon), PSD95 (post-synaptic density protein), Myo7A (hair cells), DAPI (nucleus).

FIGS. 6A-6D ABR thresholds and DPOAEs after moderate noise trauma among control (n=9) versus losartan rescue treatment (n=9) versus losartan pre-treatment (n=9) mice up to 30 days post-noise exposure at (A) 8 kHz, (B) 16 kHz, (C) and 32 kHz.

FIG. 7 Odds for incident hearing loss, tinnitus or cochlear implant.

FIG. 8. Female stratification of cohorts. Forest plot illustrates odds-ratios and 95% confidence intervals for hearing loss, tinnitus, and cochlear implantation for each pair-wise combination of anti-hypertensive classes. PSM=propensity score matching. ARB=angiotensin receptor blocker. ACE-I=angiotensin converting enzyme inhibitor. CCB=calcium channel blocker.

FIG. 9. Male stratification of cohorts. Forest plot illustrates odds-ratios and 95% confidence intervals for hearing loss, tinnitus, and cochlear implantation for each pair-wise combination of anti-hypertensive classes. PSM=propensity score matching. ARB=angiotensin receptor blocker. ACE-I=angiotensin converting enzyme inhibitor. CCB=calcium channel blocker.

FIGS. 10A-10C. Confocal images of the tunnel of Corti from a transgenic mouse in which GFP has been coupled to the promoter driving Agt2r expression. These images reveal expression of Agt2r in cells supporting the outer hair cells more so than the inner hair cells, possibly Dieter cells, that maintain endocochlear potential and calcium recycling needed to normal hair cell functioning.

FIG. 11. AT2R blockade by continuous subcutaneous infusion of PD 123-319, a selective AT2R antagonist, potentiates noise-induced dysfunction in CBA/CaJ mice. * p<0.05. n=8-10 per group.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Auditory disorders, including hearing loss, tinnitus, and Meniere's Disease, represent a significant global health challenge, affecting millions of individuals and often leading to debilitating symptoms such as impaired communication, chronic discomfort, and reduced quality of life. Despite the prevalence of these conditions, there are currently no FDA-approved pharmacological treatments specifically targeting their underlying pathophysiology. Conventional approaches primarily rely on supportive management, such as hearing aids, cochlear implants, or surgical interventions, which are invasive, costly, and often fail to address the root causes of these disorders. Off-label use of medications has shown limited efficacy, leaving patients with poorly controlled symptoms and significant functional impairments. Furthermore, the mechanisms driving auditory disorders, such as inflammation, vascular dysfunction, and oxidative stress, remain inadequately targeted by existing therapies.

The present disclosure addresses these limitations by repurposing medications that target the renin-angiotensin system (RAS), including angiotensin receptor blockers (ARBs), calcium channel blockers (CCBs), angiotensin II type 2 receptor (AT2R) agonists, and Mas receptor (MasR) agonists, for the prevention and treatment of auditory disorders. These drugs, traditionally used for hypertension, chronic kidney disease, and heart failure, have well-documented safety profiles and are widely available at low cost. The described approach leverages the anti-inflammatory, vasoprotective, and antioxidant properties of these medications to mitigate the pathophysiological processes underlying auditory disorders. Preclinical studies have demonstrated that ARBs, such as losartan, can protect against noise-induced hearing loss, preserve cochlear synapses, and reduce auditory brainstem response (ABR) threshold shifts. Additionally, retrospective analyses of patient data reveal significant associations between RAS-targeting drugs and reduced incidence of hearing loss, tinnitus, and cochlear implantation.

By introducing a novel pharmacological approach that repurposes existing medications, the described technology provides a transformative solution to the unmet medical need for effective treatments for auditory disorders. The methods disclosed herein enable both preventive and rescue strategies, offering patients safe, accessible, and clinically impactful options to address conditions that have long been underserved by conventional therapies.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 16th Edition, by M. Larranga, R Lewis Sr., and R Lewis, New York, N.Y., 2016.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, an “effective amount” means an amount sufficient to produce a selected effect, such as alleviating symptoms of a disease or disorder. The term to “treat,” as used herein, means reducing the frequency with which symptoms are experienced by a patient or subject or administering an agent or compound to reduce the frequency with which symptoms are experienced.

The term “delivery vehicle” or “carrier” refers to any kind of device or material which can be used to deliver the invention in vivo.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

As used herein “injecting, administering or applying” includes administration of the invention by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, ophthalmic, or pulmonary.

As used herein, a “subject in need thereof” is a patient, animal (domestic (cat, dog) or farm animal (livestock, horse, cow)), mammal, or human, who will benefit from the method of this invention.

A disease, condition, or disorder is “alleviated” if the severity of a symptom of the disease or disorder, the frequency with which such a symptom is experienced by a subject, or both, are reduced.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

Methods involving conventional molecular biology techniques are described herein. Such techniques are generally known in the art and are described in detail in methodology treatises, such as Molecular Cloning: A Laboratory Manual, 4th ed., Green and Sambrook, Cold Spring Harbor Laboratory Press, Cold Spring Harbor, N.Y., 2014; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981.

### Overview of Hearing

Hearing, or auditory perception, is the ability to perceive sounds through an organ, such as an ear, by detecting vibrations as periodic changes in the pressure of a surrounding medium. The academic field concerned with hearing is auditory science.

There are three main components of the human auditory system: the outer ear, the middle ear, and the inner ear.

The outer ear includes the pinna, the visible part of the ear, as well as the ear canal, which terminates at the eardrum, also called the tympanic membrane. The pinna serves to focus sound waves through the ear canal toward the eardrum. Because of the asymmetrical character of the outer ear of most mammals, sound is filtered differently on its way into the ear depending on the location of its origin. This gives these animals the ability to localize sound vertically. The eardrum is an airtight membrane, and when sound waves arrive there, they cause it to vibrate following the waveform of the sound. Cerumen (ear wax) is produced by ceruminous and sebaceous glands in the skin of the human ear canal, protecting the ear canal and tympanic membrane from physical damage and microbial invasion.

The middle ear consists of a small air-filled chamber that is located medial to the eardrum. Within this chamber are the three smallest bones in the body, known collectively as the ossicles which include the malleus, incus, and stapes (also known as the hammer, anvil, and stirrup, respectively). They aid in the transmission of the vibrations from the eardrum into the inner ear, the cochlea. The purpose of the middle ear ossicles is to overcome the impedance mismatch between air waves and cochlear waves, by providing impedance matching.

Also located in the middle ear are the stapedius muscle and tensor tympani muscle, which protect the hearing mechanism through a stiffening reflex. The stapes transmits sound waves to the inner ear through the oval window, a flexible membrane separating the air-filled middle ear from the fluid-filled inner ear. The round window, another flexible membrane, allows for the smooth displacement of the inner ear fluid caused by the entering sound waves.

The inner ear consists of the cochlea, which is a spiral-shaped, fluid-filled tube. It is divided lengthwise by the organ of Corti, which is the main organ of mechanical to neural transduction. Inside the organ of Corti is the basilar membrane, a structure that vibrates when waves from the middle ear propagate through the cochlear fluid-endolymph. The basilar membrane is tonotopic, so that each frequency has a characteristic place of resonance along it. Characteristic frequencies are high at the basal entrance to the cochlea, and low at the apex. Basilar membrane motion causes depolarization of the hair cells, specialized auditory receptors located within the organ of Corti. [6] While the hair cells do not produce action potentials themselves, they release neurotransmitter at synapses with the fibers of the auditory nerve, which does produce action potentials. In this way, the patterns of oscillations on the basilar membrane are converted to spatiotemporal patterns of firings which transmit information about the sound to the brainstem.

The sound information from the cochlea travels via the auditory nerve to the cochlear nucleus in the brainstem. From there, the signals are projected to the inferior colliculus in the midbrain tectum. The inferior colliculus integrates auditory input with limited input from other parts of the brain and is involved in subconscious reflexes such as the auditory startle response.

The inferior colliculus in turn projects to the medial geniculate nucleus, a part of the thalamus where sound information is relayed to the primary auditory cortex in the temporal lobe.

Sound is believed to first become consciously experienced at the primary auditory cortex. Around the primary auditory cortex lies Wernicke's area, a cortical area involved in interpreting sounds that is necessary to understand spoken words.

Disturbances (such as stroke or trauma) at any of these levels can cause hearing problems, especially if the disturbance is bilateral. In some instances, it can also lead to auditory hallucinations or more complex difficulties in perceiving sound.

### Diseases/Disorders

### Hearing Loss

Despite regulations defining “unsafe” levels of noise intensity and duration, the incidences of noise-induced hearing loss (NIHL) continue to be high in both adults and children globally. Cumulative exposure to sound levels of 85 dB SPL (e.g. food blender) and above is harmful to hearing, with risk increasing as intensity and duration arise. Exposure to even very brief (sub-second) sound levels at or above 120 dB SPL (e.g. gunshot) can result in permanent threshold shifts in hearing.

The pathophysiology of NIHL is insidious, following a course of initial injury to more vulnerable portions of the acoustic system (cochlear synapses), later loss of hair cells and neurons, and later still, clinically significant and progressive hearing loss. Noise exposure causes oxidative damage and accumulation of reactive oxygen species (ROS), it also induces an immune response, including activation of NF kB signaling and upregulation of pro-inflammatory cytokines such as IL6, TNF-α, and TGF-β, and other inflammatory mediators including COX2. The resulting recruitment of monocytes and macrophages and further secretion of cytokines and growth factors create an environment of excess inflammation, leading to microcirculation destruction, tissue damage, and hearing loss. Molecules that interrupt the inflammatory cascade by inhibiting TGF-β³⁵, NF-kB and IL-1β, or TNF-α protect against noise damage, thus providing support for the causal role that inflammation plays in NIHL. For example, metformin was recently shown to protect against ABR shifts and lessen hair cell loss in response to noise trauma. Ang II is a major player in inflammatory processes of multiple organ systems, including in cardiovascular, renal, hepatic, and retinal pathologies. These various end-organ protective effects have motivated early studies exploring the impact of AT1R blockers in hearing loss. AT1R blockade via losartan attenuated diabetic otopathy in Zucker rats. In a preclinical model of vestibular schwannoma, AT1R blockade with losartan resulted in preservation of hearing and normalization of the extracellular matrix contents and vascular distribution and decreased IL-6/STAT3 signaling. Recently, AT1R blockade using telmisartan was found to protect against aminoglycoside-induced hearing loss, with preservation of hair cell counts.

Hearing loss occurs when there is reduced ability to detect and/or encode sound waves in the ear and/or disrupted perception by the brain. Hearing loss affects people of all ages and can be caused by many different factors. The three basic categories of hearing loss are sensorineural hearing loss, conductive hearing loss and mixed hearing loss. Recently, the term of Aural Diversity has come into greater use, to communicate hearing loss and differences in a less negatively associated term.

### Sensorineural Hearing Loss

This type of hearing loss occurs when the inner ear or the actual hearing nerve itself becomes damaged. This loss generally occurs when some or all of the hair cells or their synapses within the cochlea are damaged.

Sensorineural loss is the most common type of hearing loss. It can be a result of aging, exposure to loud noise, injury, disease, infection, inflammation, autoimmune reaction, neoplasms, certain drugs, and inherited condition, or for reasons that are not understood (idiopathic). This type of hearing loss is typically not medically treatable; however, many people with mild, moderate, or severe levels of this type of loss find that hearing aids can be beneficial. For profound hearing loss, hearing restoration can be achieved by cochlear implantation.

### Sudden Sensorineural Hearing Loss

Sudden sensorineural hearing loss may occur very suddenly or over the course of a few days

### Conductive Hearing Loss

This type of hearing loss occurs in the outer or middle ear where sound waves are not able to carry all the way through to the inner ear. Sound may be blocked by earwax or a foreign object located in the ear canal; the middle ear space may be impacted with fluid, infection, scarring, an abnormal collection of skin (cholesteatoma), or a bone abnormality; or the eardrum may have been injured.

### Mixed Hearing Loss

Sometimes people can have a combination of both sensorineural and conductive hearing loss. They may have a sensorineural hearing loss and then develop a conductive component in addition.

### There are Defined Degrees of Hearing Loss:

Mild hearing loss—People with mild hearing loss have difficulties keeping up with conversations, especially in noisy surroundings. The quietest sounds that people with mild hearing loss can hear with their better ear are between 25- and 40-dB HL.
Moderate hearing loss—People with moderate hearing loss have difficulty keeping up with conversations when they are not using a hearing aid. On average, the quietest sounds heard by people with moderate hearing loss with their better ear are between 40- and 70-dB HL.
Severe hearing loss—People with severe hearing loss depend on powerful hearing aid. However, they often rely on lip-reading even when they are using hearing aids. The quietest sounds heard by people with severe hearing loss with their better ear are between 70- and 95-dB HL.
Profound hearing loss—People with profound hearing loss are very hard of hearing and they mostly rely on lip-reading and sign language. The quietest sounds heard by people with profound hearing loss with their better ear are from 95 dB HL or more.

Hearing loss can be attributed to many different aspects including noise-induced hearing loss, acoustic trauma, presbycusis, age-related hearing loss, post-infectious hearing loss, idiopathic hearing loss, autoimmune hearing loss, tumor-related hearing loss, and/or drug-induced hearing loss (ototoxicity).

### Endolymphatic Hydrops/Meniere's Disease

Endolymphatic hydrops is a condition in which the electrolyte concentrations and related pressures within the fluid compartments of the inner ear become disrupted, resulting in temporary or permanent sensorineural hearing loss, tinnitus, vertigo, and a sensation of ear fullness. When this process becomes recurrent and progressive, the patient is diagnosed with Ménière's disease. Ménière's disease is a disorder of the inner ear that causes severe and disabling dizziness (vertigo), ringing in the ears (tinnitus), hearing loss, and a feeling of fullness or congestion in the ear. Ménière's disease usually affects only one ear, but in 15% to 25% of people with the disorder, both ears may be affected. The symptoms of Ménière's disease are associated with a fluid imbalance in a part of the inner ear called the labyrinth.

Attacks of dizziness may come on suddenly or after a short period of tinnitus or muffled hearing. Some people have single attacks of dizziness separated by long periods of time. Others may experience many attacks close together over several days. Some people with Ménière's disease have vertigo so extreme that they lose their balance and fall. These episodes are called “drop attacks.”

According to the American Academy of Otolaryngology-Head and Neck Surgery, Ménière's disease can develop at any age, but it is more likely to occur in adults between 40 and 60 years of age. Approximately 615,000 individuals in the United States have Ménière's disease, and about 45,500 cases are newly diagnosed each year, according to the American Hearing Research Foundation.

### Tinnitus

Subjective tinnitus is the perception of sound in the ears when no external sound is present. It's often described as a ringing in the ears, but it can also sound like buzzing, roaring, chirping, clicking, hissing, humming, whistling, or sizzling. Objective tinnitus is the perception of sound in the patient's ears that can also be heard by others, such as with the use of a stethoscope, that is caused by an internal process, such as heart beating, blood passing through vessels, or the twitching of muscles connected to the ossicles or middle ear.

Tinnitus is a symptom of an issue with the auditory system, which includes the brain and ears. It's common, affecting 10-25% of adults, and can also occur in children.

### Hearing Test

Hearing can be measured by behavioral tests using an audiometer. Electrophysiological tests of hearing can provide accurate measurements of hearing thresholds even in unconscious subjects. Such tests include auditory brainstem evoked potentials (ABR), otoacoustic emissions (OAE) and electrocochleography (ECochG). Technical advances in these tests have allowed hearing screening for infants to become widespread.

Hearing can be measured by mobile applications which includes audiological hearing test function or hearing aid application. These applications allow the user to measure hearing thresholds at different frequencies (audiogram). Despite possible errors in measurements, hearing loss can be detected.

### Treatment

Provided herein is a prevention and ‘rescue dosing’/treatment strategy wherein pre and/or post-trauma, such as noise exposure, administration of, for example, losartan is nearly equally protective against preventing and treating hearing loss in response to acoustic trauma.

For example, provide herein is the use of RAS drugs to treat after the onset of an auditory disorder, in this case hearing loss. Such a rescue treatment strategy is of clinical relevance for patients with an auditory disorder, including those who unexpectedly encounter an auditory affront, such as a loud noise exposure, an ototoxic drug exposure, the sudden onset of hearing loss (i.e. SSNHL) or tinnitus, or who begin to experience Meneire's-type vertigo and hearing loss attacks, to provide just a few examples.

Further, auditory disorders may be effectively treated using compounds that act through other receptors, such as angiotensin II type 2 receptors (AT2R) agonists (e.g., buloxibutid or C21) or Mas receptor agonists (e.g., angiotensin-(1-7) or AVE0779). This is specifically suggested by the data in the retrospective review of data from hypertensive patients showing that angiotensin receptor blockers (ARBs) are associated with a significantly larger protection against the development of auditory disorders than are angiotensin converting enzyme inhibitors (ACE-Is). This suggests that the inhibition of angiotensin II production that is induced during ACE-I therapy has potential negative downstream effects by limiting the activation of these alternative RAS receptors. In the case of ARB therapy, angiotensin II is still produced and is free to bind to the AT2R, which is known to have anti-inflammatory, antioxidant, vasoprotective effects in other organ systems. Furthermore, metabolism of angiotensin II to angiotensin-(1-7) makes this molecule available to bind to the Mas receptor, which is also known to have profound renoprotective, cardioprotective, and neuroprotective effects. By extension, the data suggest that drugs or compounds targeting these receptors are likely to produce protective effects against auditory disorders. Drugs or other compounds that target these compounds are not currently FDA approved for use in humans, but a candidate AT2R agonist, buloxibutid, is currently in stage III clinical trials for use in pulmonary fibrosis, with patents for its use in this and related disorders.

Examples of ARBs for use herein include, but are not limited to, Azilsartan (Edarbi), Losartan (Cozaar), Valsartan (Diovan), Candesartan (Atacand), Telmisartan (Micardis), Irbesartan (Avapro), Olmesartan (Benicar), Fimasartan (Kanarb), Saprisartan, and Eprosartan (Teveten). In one embodiment, the ARB is not telmesartan. In another embodiment, the drug/compound used herein is not a peroxisome proliferator activated receptor γ (PPAR γ) agonist.

Also, of use in the invention are AT2R agonists, including, but not limited to, buloxibutid, β-Pro7Ang III, angiotensin-(1-7), alamandine, and [Sar1,Ile8] Angiotensin II.

Of use in the invention are calcium channel blockers (CBBs), angiotensin converting enzyme (ACE) inhibitors, and/or Mas receptor (MasR) agonists.

Calcium channel blockers (CBBs) include, but are not limited to, Amlodipine (Norvasc), Aranidipine (Sapresta), Azelnidipine (Calblock), Barnidipine (HypoCa), Benidipine (Coniel), Cilnidipine (Atelec, Cinalong, Siscard), Clevidipine (Cleviprex), Efonidipine (Landel), Felodipine (Plendil), Isradipine (DynaCirc, Prescal), Lacidipine (Motens, Lacipil), Lercanidipine (Zanidip), Manidipine (Calslot, Madipine), Nicardipine (Cardene, Carden SR), Nifedipine (Procardia, Adalat), Nilvadipine (Nivadil), Nimodipine (Nimotop), Nisoldipine (Baymycard, Sular, Syscor), Nitrendipine (Cardif, Nitrepin, Baylotensin), Pranidipine (Acalas), Fendiline, Gallopamil, Verapamil (Calan, Isoptin), Diltiazem (Cardizem), Mibefradil, Bepridil, Flunarizine, Fluspirilene, Gabapentin, Pregabalin, and Ziconotide.

Angiotensin converting enzyme (ACE) inhibitors include, but are not limited to, Captopril, Enalapril, Lisinopril, Benazepril, Fosinopril, Quinapril, Ramipril, Perindopril, Moexipril, Trandolapril, Imidapril, Cilazapril, and Ceronapril.

MasR agonists include, but are not limited to, Angiotensin-(1-7), [D-Ala7]-Angiotensin-(1-7), p-Amino-phenylalanine-containing analogues of Angiotensin-(1-7), HPBCD-Angiotensin-(1-7), cyclodextran-bound angiotensin-(1-7), BIO101, CGEN-856S, CGEN-857, AVE0779, and CGP42112 or combinations thereof.

In various embodiments, the method to prevent or treat an auditory disorder comprises administering to a subject in need thereof an angiotensin receptor blocker (ARB), an angiotensin converting enzyme (ACE) inhibitor, an angiotensin II type 2 receptor (AT2R) agonist, a Mas receptor (MasR) agonist, or a combination thereof, wherein the ARB is not telmisartan. In one embodiment, the ARB may be selected from Azilsartan, Losartan, Valsartan, Candesartan, Irbesartan, Olmesartan, Fimasartan, Saprisartan, Eprosartan, or combinations thereof, and may be administered orally, intravenously, or via a film or coating on an implantable device such as a cochlear implant or tympanostomy tube. In another embodiment, the ACE inhibitor may be selected from Captopril, Enalapril, Lisinopril, Benazepril, Fosinopril, Quinapril, Ramipril, Perindopril, Moexipril, Trandolapril, Imidapril, Cilazapril, or Ceronapril, and may be administered systemically or locally. In a further embodiment, the AT2R agonist may be buloxibutid, Compound 21, M024, CGP42112, PD-123-319, β-Pro7Ang III, angiotensin-(1-7), alamandine, or [Sar1,Ile8] Angiotensin II, and the MasR agonist may be Angiotensin-(1-7), [D-Ala7]-Angiotensin-(1-7), p-Amino-phenylalanine-containing analogues of Angiotensin-(1-7), HPβCD-Angiotensin-(1-7), cyclodextran-bound angiotensin-(1-7), BIO101, CGEN-856S, CGEN-857, AVE0779, or CGP42112, with administration routes including oral, injectable, transdermal, or as a coating on an implantable device. The administration may occur hours, days, weeks, months, or years prior to or after the onset of the auditory disorder, and may be performed as a single dose, repeated daily, or in cycles, depending on the subject's condition and therapeutic needs. The subject may be a human or animal, with or without hypertension, and the treatment may be used alone or in combination with other therapeutics such as steroids.

In different embodiments, the method to prevent or treat tinnitus, Meniere's Disease, or dizziness/vertigo comprises administering to a subject in need thereof an angiotensin receptor blocker (ARB), a calcium channel blocker (CCB), an angiotensin converting enzyme (ACE) inhibitor, an angiotensin II type 2 receptor (AT2R) agonist, a Mas receptor (MasR) agonist, or a combination thereof. In one embodiment, the ARB may be selected from Azilsartan, Losartan, Valsartan, Candesartan, Irbesartan, Olmesartan, Fimasartan, Saprisartan, Eprosartan, or combinations thereof, and may be administered orally, intravenously, or as a film or coating on an implantable device such as a cochlear implant or tympanostomy tube. In another embodiment, the CCB may be selected from Amlodipine, Felodipine, Nifedipine, Verapamil, Diltiazem, or other listed CCBs, and may be administered systemically or locally. In a further embodiment, the ACE inhibitor may be selected from Captopril, Enalapril, Lisinopril, Benazepril, Fosinopril, Quinapril, Ramipril, Perindopril, Moexipril, Trandolapril, Imidapril, Cilazapril, or Ceronapril, and may be administered by oral, injectable, or topical routes. The AT2R agonist may be buloxibutid, Compound 21, M024, CGP42112, PD-123-319, β-Pro7Ang III, angiotensin-(1-7), alamandine, or [Sar1,Ile8] Angiotensin II, and the MasR agonist may be Angiotensin-(1-7), [D-Ala7]-Angiotensin-(1-7), p-Amino-phenylalanine-containing analogues of Angiotensin-(1-7), HPβCD-Angiotensin-(1-7), cyclodextran-bound angiotensin-(1-7), BIO101, CGEN-856S, CGEN-857, AVE0779, or CGP42112, with administration routes including oral, injectable, transdermal, or as a coating on an implantable device. The administration may be performed hours, days, weeks, months, or years prior to or after the onset of tinnitus, Meniere's Disease, or dizziness/vertigo, and may be performed as a single dose, repeated daily, or in cycles, depending on the subject's condition and therapeutic needs. The subject may be a human or animal, with or without hypertension, and the treatment may be used alone or in combination with other therapeutics such as steroids.

In various embodiments, the method to prevent or treat a shift in auditory brainstem response (ABR) from noise induced trauma comprises administering to a subject in need thereof an angiotensin receptor blocker (ARB), an angiotensin converting enzyme inhibitor (ACEi), an angiotensin II type 2 receptor (AT2R) agonist, a Mas receptor agonist, or a combination thereof. In one embodiment, the ARB may be selected from Azilsartan, Losartan, Valsartan, Candesartan, Irbesartan, Olmesartan, Fimasartan, Saprisartan, Eprosartan, or combinations thereof, and may be administered orally, intravenously, or as a film or coating on an implantable device such as a cochlear implant or tympanostomy tube. In another embodiment, the ACE inhibitor may be selected from Captopril, Enalapril, Lisinopril, Benazepril, Fosinopril, Quinapril, Ramipril, Perindopril, Moexipril, Trandolapril, Imidapril, Cilazapril, or Ceronapril, and may be administered systemically or locally. In a further embodiment, the AT2R agonist may be buloxibutid, Compound 21, M024, CGP42112, PD-123-319, β-Pro7Ang III, angiotensin-(1-7), alamandine, or [Sar1,Ile8] Angiotensin II, and the Mas receptor agonist may be Angiotensin-(1-7), [D-Ala7]-Angiotensin-(1-7), p-Amino-phenylalanine-containing analogues of Angiotensin-(1-7), HPβCD-Angiotensin-(1-7), cyclodextran-bound angiotensin-(1-7), BIO101, CGEN-856S, CGEN-857, AVE0779, or CGP42112, with administration routes including oral, injectable, transdermal, or as a coating on an implantable device. The administration may occur hours, days, weeks, months, or years prior to or after the onset of noise induced trauma, and may be performed as a single dose, repeated daily, or in cycles, depending on the subject's condition and therapeutic needs. The subject may be a human or animal, with or without hypertension, and the treatment may be used alone or in combination with other therapeutics such as steroids.

### Administration

Provided herein is the discovery that medications that target the renin angiotensin system (RAS), typically used to treat hypertension, have remarkable effects at protecting against hearing loss, sudden sensorineural hearing loss, tinnitus, and Meniere's Disease. Preclinical studies have revealed that daily oral consumption of anti-hypertensive dosages of losartan, an angiotensin receptor blocker, can both prevent and rescue hearing function in response to noise trauma in mice. In a large retrospective review of patient data, large, clinically significant associations of fewer diagnoses of hearing loss, tinnitus, cochlear implantation, and Meniere's Disease have been identified among patients on treatment. This discovery suggests that medications, such as losartan, can be prescribed at typical dosages for treating hypertension or chronic kidney disease among existing patient populations at risk for auditory disorders. Further provided herein is the treatment of individuals who develop such disorders with the administration of these medications using a rescue strategy. In repurposing RAS drugs for the prevention and treatment of auditory disorders, patients will have made available safe, effective, and low-cost options with few barriers to immediate clinical implementation.

Compounds/drugs/compositions for use in the invention can be administered to the subject once. Alternatively, such compounds can be administered once or twice daily to a subject in need thereof for a period of from about two to about twenty-eight days, or from about seven to about ten days. Such compounds can also be administered once or twice daily to a subject for a period of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 times per year, or a combination thereof. Treatment can be carried out for weeks, months or even years, especially in the case of age-related hearing loss and Meniere's disease/endolymphatic hydrops, which are chronic and progressive over a lifetime. In some aspects, the administration is oral. In some aspects, the administration is carried out hours (e.g., 1, 2, 3, 4, 5, 6, 7, 8, 9, 10, 11, 12, 13, 14, 15, 16, 17, 18, 29, 20, 21, 22, 23, or 24 hours), days (e.g., 1, 2, 3, 4, 5, 6 or 7 days) or weeks (e.g., 1, 2, 3, 4, 5, 6, 7, 8, 9, 10, 11, 12 or more weeks) prior to or after the onset of an auditory disorder. Treatment can be carried out for weeks, months or even years after the onset of an auditory disorder. Furthermore, such compounds can be co-administrated with another therapeutic, such as a steroid.

The compounds can be administered to a subject by any means suitable for delivering the compounds to the subject. The compounds/drugs/compositions can be formulated and administered to reduce the symptoms associated a hearing loss, tinnitus or Meniere's Disease of a subject, such as a human or animal (e.g., a dog, cat, or horse). They can be administered by any conventional means available for use in conjunction with pharmaceuticals, either as individual therapeutic active ingredients or in a combination of therapeutic active ingredients. They can be administered alone but are generally administered with a pharmaceutical carrier selected on the basis of the chosen route of administration and standard pharmaceutical practice.

A therapeutically effective dose of compounds/drugs/compositions can depend upon a number of factors known to those of ordinary skill in the art. The dose(s) of the compounds/drugs/compositions can vary, for example, depending upon the identity, size, and condition of the subject or sample being treated, further depending upon the route by which the compound/drug/composition is to be administered. These amounts can be readily determined by a skilled artisan. Any of the therapeutic applications described herein can be applied to any subject in need of such therapy, including, for example, a mammal such as a dog, a cat, a cow, a horse, a rabbit, a monkey, a pig, a sheep, a goat, or a human.

Pharmaceutical compositions for use in accordance with the invention can be formulated in conventional manner using one or more physiologically acceptable carriers or excipients. The therapeutic compositions of the invention can be formulated for a variety of routes of administration, including systemic, oral, topical or localized administration. Techniques and formulations generally can be found in Remmington's Pharmaceutical Sciences, Meade Publishing Co., Easton, Pa. (20^{th} Ed., 2000), the entire disclosure of which is herein incorporated by reference. For systemic administration, an injection is useful, including intramuscular, intravenous, intraperitoneal, intratympanic, and subcutaneous. For injection, the therapeutic compositions of the invention can be formulated in liquid solutions, for example in physiologically compatible buffers such as Hank's solution or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

The pharmaceutical compound/drug/composition can be impregnated in or on a thin film or used as a coating, the film and/or coating can be applied to the surface of an implantable device, such as tympanostomy tubes, ossicular chain prostheses, silastic buttons, cochlear implants, or other middle or inner ear implants.

A pharmaceutical compound/drug/composition can be administered in conjunction with a pharmaceutically acceptable carrier, for any of the therapeutic effects discussed herein. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents/drugs.

Sterile injectable solutions can be prepared by incorporating the compound/drugs in the required amount in an appropriate solvent with one or a combination of ingredients enumerated herein, as required, followed by filtered sterilization. Generally, dispersions are prepared by incorporating the active compound into a sterile vehicle which contains a dispersion medium. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

In some embodiments, the drug/compound can be applied via transdermal delivery systems, which slowly releases the active compound for percutaneous absorption. Permeation enhancers can be used to facilitate transdermal penetration of the active factors in the conditioned media. Transdermal patches are described in for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475.

### EXAMPLES

The disclosure can be better understood by reference to the following examples which are offered by way of illustration. The disclosure is not limited to the examples given herein.

### Example I

### Hearing Preservation by Angiotensin Receptor Blockade in Murine Acoustic Trauma and in Hypertensive Patients

### Overview:

Losartan provides hearing protection among hypertensive patients and from noise-induced hearing loss in mice.

### Background

Hypertension and noise trauma impair cochlear microcirculation through multiple overlapping mechanisms resulting in progressive cellular damage and hearing loss. Losartan is an angiotensin II type 1 receptor antagonist (ARB) that has anti-inflammatory and vascular modulating effects. Losartan has well described vasoprotective effects in microvascular diseases with emerging evidence for auditory protection.

### Methods

Hearing outcomes among patients with hypertension taking different first-line anti-hypertensive regimens were compared using a large electronic health records database to look for a potential association with ARBs and hearing protection. In a parallel preclinical study, CBA/J mice (age 9-10 weeks, n=5-7 per group) were randomized to standard or losartan-infused chow (20 mg/kg/day) starting three days prior to mild (temporary threshold shift) or moderate (permanent threshold shift) intensity noise exposure. Auditory brainstem responses and distortion product otoacoustic emissions were recorded for 14 days after noise exposure. Cochleae were collected for whole-mount and immunofluorescent imaging to quantify inner and outer hair cell (IHC) survival and synapse damage.

### Results

After propensity score matching to balance for common covariates that associate with sensorineural hearing loss, both angiotensin receptor blockers (OR 0.73, 0.72-0.74, p<0.0001) and calcium channel blockers (OR 0.71, 0.70-0.72, p<0.0001) demonstrated hearing protective associations versus thiazide diuretics in patients with hypertension. Similar protective associations were observed with tinnitus and cochlear implantation diagnoses. In mice, treatment with losartan blunted temporary threshold shifts in ABR recordings recorded 1 day after mild-intensity noise exposure. Losartan treatment also preserved IHC synapse counts relative to the control group (15.3 vs 19.9 synapses/IHC, p=0.001). In mice experiencing larger ABR threshold shifts after moderate-intensity noise, losartan blunted ABR threshold increases at 1-day post-exposure, which persisted to 14 days with nearly complete protection against the permanent threshold shifts observed in the control group. Additionally, losartan resulted in significantly improved ABR wave 1 amplitudes and latencies at 14 days.

### Conclusions

Angiotensin receptor blockade is associated with decreased odds of sensorineural hearing loss, tinnitus, and cochlear implant diagnoses in hypertensive patients as compared to other first-line antihypertensive treatment. Furthermore, losartan treatment in mice prevents both temporary and permanent ABR threshold shifts after noise overexposure, possibly by exerting a protective effect against noise-induced cochlear synaptopathy.

### INTRODUCTION

Two-thirds of older adults suffer from hearing loss (1), a condition that associates with higher rates of hospitalizations, frailty, dementia, and neuropsychiatric disorders (2, 3). In the majority of adult patients with hearing loss, this is attributable to breakdown of signaling within the inner ear, which houses the cochlear inner hair cells (IHCs) and their synaptic connections to the spiral ganglion neurons (SGNs). Axons from the SGNs combine to form the auditory nerve, which relays acoustic information to the higher auditory processing centers. Hearing loss that results from disruption of signal transmission at one or more points within these components of the auditory pathway is termed sensorineural hearing loss (SNHL). Though SNHL is often attributed simply to aging, the variable effect of age on acoustic function is modulated by other factors, such as an individual's accumulated noise exposure and comorbid metabolic disorders, including hypertension. Noise pollution continues to be a pervasive public health hazard, with audiometric evidence of noise-induced hearing loss (NIHL) in ~1 in 4 adults with measurable hearing loss and, surprisingly, 1 in 5 adults without clinically significant hearing loss (4, 5). Aside from high dose steroids which can be prescribed to rescue hearing for patients with idiopathic sudden SNHL (6-8), there are currently no definitive preventive or rescue clinical treatments for SNHL. Management of hypertension can be protective against hearing declines.

Progressive SNHL is compounded by diseases that drive systemic metabolic dysregulation and microvascular damage, such as hypertension, obesity, type-2 diabetes, and hyperlipidemia (9-16). The microvascular pathophysiologic processes that precipitate hearing loss in the metabolic syndrome mechanistically overlap with those that occur after loud noise exposure, contributing to enhanced risk of hearing loss after occupational noise exposure for people with pre-existing hypertension (17-19).

Even in the absence of hypertension, noise overexposure exerts a chronic inflammatory response leading to deleterious effects on hearing over time (20). At the cochlear synapses between ear hair cells and afferent type 1 SGNs, also known as ribbon synapses, these sublethal changes persist and accelerate presbycusis (21). Synaptopathy often precedes eventual neuronal degeneration and development of clinically apparent progressive SNHL, in some cases by many years (21-24). The harmful effects of acoustic trauma arise via vasculopathic (25-27), cellular excitotoxic (28, 29), and inter-related inflammatory (22-24, 30-33) mechanisms. These overlap with the pathologic processes described in age-related hearing loss, which include immune cell infiltration, blood-labyrinth barrier disruption via impaired strial circulation, and subsequent derangement of the endocochlear potential that is required for normal hair cell signaling (28, 24). Given the multifactorial and overlapping pathophysiology of noise-induced and hypertension-associated hearing loss, a potential pharmaceutical therapeutic that interrupts overlapping mechanisms of acoustic trauma and microvascular disease should have a higher likelihood of demonstrating efficacy.

To this end, drugs targeting the renin angiotensin system represent promising candidates for hearing protection. The renin angiotensin aldosterone pathway has conventionally been shown to play a role in multiple chronic disease states including hypertension (35), renal dysfunction (35, 36), pulmonary disease (37), and neurovascular disease (38, 39). Inhibitors of the renin angiotensin system, such as angiotensin converting enzyme inhibitors (ACEi) and angiotensin II type 1 receptor blockers (ARBs), are widely utilized to treat patients with cardiovascular and renal disease and have well documented effects in reducing inflammation, oxidative stress, vascular ischemia, and subsequent fibrosis in multiple organ systems (35, 40, 41). As such, ARBs are among first-line drugs for hypertension and are prescribed prophylactically to newly diagnosed type-2 diabetics to reduce the risk of diabetic nephropathy (42, 43). These protective actions may result from modulation of inter-connected inflammatory, fibrotic, and vasculopathic cascades, including TNF-alpha, NF-kB, and TGF-beta and their down-stream effector immune cells (39-41, 44-46). In recent preclinical studies, ARBs have demonstrated otoprotection against aminoglycoside ototoxicity (47, 48), Alport-syndrome related oto/nephropathy (47-49), and diabetic otopathy (50). An intriguing study of vestibular schwannoma in both mice and human patients suggests a role for losartan in decreasing edema, inflammation, and preventing hearing loss associated with the vestibular schwannoma microenvironment (51, 52), however the hearing preservation effect in humans remains controversial (53).

With these beneficial and multifactorial effects of ARB therapy in mind, it was hypothesized that hypertensive patients treated with losartan would have a lower incidence of SNHL and related hearing diagnostic codes (tinnitus, cochlear implantation), compared to other first-line anti-hypertensive drugs using a large electronic health records (EHR) database. The hypothesis that angiotensin receptor blockade via losartan is protective against noise-induced hearing loss and cochlear synaptopathy after mild and moderate acoustic trauma in mice was tested.

### Materials and Methods

### Ethical Approvals

This study was reviewed by The University of Iowa's Institutional Review Board and determined to be exempt and followed the Strengthening the Reporting of Observational Studies in Epidemiology (STROBE) reporting guidelines. All animal experimental protocols in this study were approved by the University of Iowa Institutional Animal Care and Use Committee (Protocol 3022519).

### Data Sources

Study queries were executed on the TriNetX Research Network multi-national electronic health records (EHR) ‘research’ database representing about 130 million patients from 92 healthcare organizations and 5 countries. TriNetX is a federated, live, cloud-based, real-world database. Queries on the database were made using Boolean operators and relative time windows on ICD-10, CPT, and other medical billing codes to define cohorts. Codes were queried from any year, though greater than 90% of all data is from after 2005.

### Query Design and Cohort Definitions

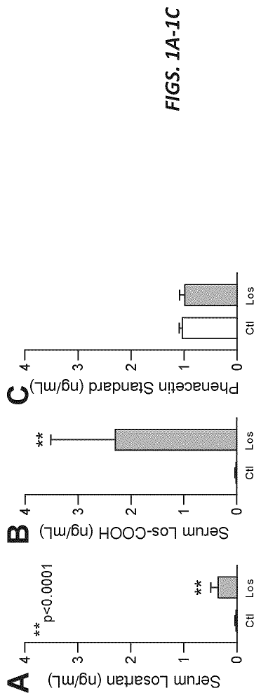

Patients any age with essential (primary) hypertension were identified using the ICD10 code I10. Using the American Academy of Family Practice guideline for hypertension management, cohorts were generated where a given cohort has a prescription for one first-line class of anti-hypertensive agent, but no other first-line anti-hypertensive agents. Anti-hypertensive classes were ARBs, angiotensin converting enzyme inhibitors (ACEs), calcium channel blockers (CCBs), and thiazide diuretics. A patient must have started the given class of anti-hypertensive within the first two years after diagnosis of essential hypertension and must have been on the anti-hypertensive drug for at least two years. Secondary analysis further sub-stratified cohorts by sex. Please see FIG. 1 for a flow diagram detailing cohort identification and Table 1 for ICD-10 codes and time-constraints for cohort definitions.

TABLE 1
ICD10 codes used to define cohorts

| Definition | ICD10/RxNorm Codes |
|---|---|
| Essential (Primary) Hypertension | |
| Time constraint: Any instance of 110 occurred within | |
| two years before, or one year after the first instance of | |
| the medication class in question. | |
| Inclusion | |
| Essential (Primary) Hypertension | I10 |
| Angiotensin Receptor Blockers (ARBs) | |
| Inclusion | |
| Losartan | 52175 |
| Valsartan | 69749 |
| Olmesartan | 321064 |

Exclusion

| All other anti-hypertensive | See other definitions |
|---|---|
| medication classes | |
| Angiotensin Converting Enzyme Inhibitors (ACEs) | |
| Inclusion | |
| Lisinopril | 29046 |
| Benazepril | 18867 |
| Enalapril | 3827 |

Exclusion

| All other anti-hypertensive | See other definitions |
|---|---|
| medication classes | |
| Calcium Channel Blockers (CCBs) | |
| Inclusion | |
| Amlodipine | 17767 |
| Felodipine | 4316 |
| Nifedipine | 7417 |

Exclusion

| All other anti-hypertensive | See other definitions |
|---|---|
| medication classes | |
| Thiazide Diuretics | |
| Inclusion | |
| Hydrochlorothiazide | 5487 |
| Indapamide | 5764 |
| Chlorthalidone | 2409 |

Exclusion

| | |
|---|---|
| All other anti-hypertensive | See other definitions |
| medication classes | |

Hypertensive Medication Longitudinal Prescription
Time constraint: Any instance of the antihypertensive
class of question occurred within two years on or after the
first instance of the medication class of question.

### Outcome Measures

Outcomes measured include hearing loss (H90-91), tinnitus (H93.1), and cochlear implantation (69930). Outcome were windowed for 0-20 years after index. Index was defined as being on the anti-hypertensive of interest for two years.

### Propensity Score Matching to Control for Confounding Covariates

Propensity score matching (PSM) is a modern statistical method to control for confounding and is an increasingly popular alternative to multi-variable logistic regression (54-56). 1:1 PSM was performed using the greedy next nearest neighbor algorithm with a caliper size of 0.1 pooled standard deviations to balance cohorts for clinical billing codes and demographic variables associated with hearing loss and general health status as outlined in Table 2.

TABLE 2
Propensity Score Matching variables

| Definition | ICD10 Code |
|---|---|
| Age-at-Index | NA |
| Female Sex | NA |
| Male Sex | NA |
| White | NA |
| Black or African American | NA |
| Asian | NA |
| Diabetes Mellitus | E08-E13 |
| Disorders of lipoprotein | E78 |
| metabolism and other lipidemias | |
| Nicotine Dependence | F17 |
| Ischemic heart disease | I20-125 |
| Cerebrovascular diseases | I60-169 |
| Chronic lower respiratory disease | J40-J47 |

### Animals and Experimental Cohorts

CBA/J mice were selected to allow for a more direct assessment of the impact of noise trauma on hearing loss due to their resistance to age-related hearing loss relative to other strains and their susceptibility to noise-induced cochlear synaptopathy relative to C57BL/6J mice (57, 58). Mixed male and female mice were divided into two experimental cohorts based on noise exposure intensity: a mild acoustic trauma that induced a temporary threshold shift (TTS) in auditory brainstem response (ABR) recordings, and a moderate acoustic trauma that induced a permanent threshold shift (PTS). Equally distributed male and female mice age 9-10 weeks were randomly assigned to receive either standard chow or losartan-infused chow (20 mg/kg/day, equivalent to ~100 mg daily dose in humans) beginning three days prior to exposure, two hours of 8-16k octave band noise at 102.5 dB (males) or 105 dB (females) for the TTS cohort (n=7 per group) or two hours of 8-16k octave band noise at 105 dB for males and 107.5 dB for females for the PTS cohort (n=6-8 per group). One mouse was excluded from the analysis after it was found that no significant threshold shift was observed after PTS noise exposure. ABR and distortion product otoacoustic emission (DPOAE) testing and serum sample collection via tail clipping was done 1 day prior to noise exposure and again at 1, 7, and 14 days after noise exposure. Mice were sacrificed 14 days after noise exposure and cochleae were collected for whole mount processing and immunohistochemical staining.

### Liquid Chromatography-Tandem Mass Spectrometry Methods

For quantification of serum levels of losartan and its pharmacologically active metabolite losartan carboxylic acid (los-COOH), liquid chromatography-tandem mass spectrometry (LC-MS/MS) was utilized as recently described (59), with minor modifications given the lower volume of serum available from mice. For extraction, 45 microliters of Acetonitrile containing 300 ng/ml phenacetin (internal standard) was added to 15 microliters of serum from each animal. After centrifugation, 20 microliters of supernatant were mixed with 40 microliters of water for analysis by LC-MS/MS (Waters Xevo TQ-S Cronos Mass Spectrometer with a Waters Acquity H-Class UPLC system). Data acquisition (Waters MassLynx 4.2) and quantitative analysis (TargetLynx) was then performed. The chromatographic peak from the total ion chromatogram was quantified by combining all multiple reaction monitoring transitions for each compound of interest (losartan, los-COOH, phenacetin).

### Auditory Brainstem Response (ABR) and Distortion Product Otoacoustic Emission (DPOAE) Recordings

Mice were anesthetized throughout ABR testing by intraperitoneal injection of ketamine/xylazine (75/10 mg/kg). Mice were maintained at consistent body temperature via heating pad. Recordings were made using subcutaneous electrodes placed just anterior-inferior to the ipsilateral ear (recording), vertex of the skull (reference), and contralateral ear (ground). ABRs were measured using the Tucker-Davis Technologies RZ6 Z-Series Bioacoustic System. Stimulus levels were measured starting from 90 dB SPL and continued down to 20 dB SPL or until no waveform was present using click stimulus and tone burst at 8-, 16-, and 32-kHz frequencies. ABR thresholds were defined as the lowest sound pressure level required to observe wave 1. The wave 1 latency and amplitude growth function were calculated in relation to the maximum wave 1 peak in response to each 16 kHz sound pressure level. DPOAEs were measured as described previously⁶⁰ with center frequencies (Fc) of 4, 8, 16, and 32 kHz. Primary signals (F1 and F2) were calculated using Fc*0.909 and Fc*1.09. F1 and F2 were generated from speakers at an intensity level from 80 to 20 dB SPL in 10 dB decrements. The level of primary tones was set equal (L1=L2) and DPOAE threshold was defined as the lowest level of F2 where DPOAE amplitude at 2F1-F2 was at least 3 dB above average level of noise floor.

### Cochlear Dissection, Whole Mounting, and Immunohistochemistry

At 14 days after noise exposure, mice were humanely euthanized and cochleae were harvested and locally perfused with 4% paraformaldehyde, post-fixed for 30 minutes with 4% paraformaldehyde at room temperature with gentle rocking, followed by two 20-minute rinses in sterile phosphate buffered saline and stored at 4° C. until dissection. The cochleae were dissected under microscopy to obtain whole mount cochlear specimens separate from the lateral wall and modiolus as described previously (61). Whole mount specimens were immunostained with antibodies against (1) C-terminal binding protein 2 (mouse anti-CtBP2 from BD Biosciences used at 1:200), (2) post-synaptic density protein 95 (mouse anti-PSD95 from DSHB used at 1:50), (3) Myosin 7A (rabbit anti-Myo7A from Proteus Biosciences used at 1:400) and appropriate secondary antibodies coupled to Alexa Fluors emission spectra. Nuclear counterstaining with Hoechst 3342, was added to further delineate individual hair cells.

### Immunofluorescent Imaging and Analysis

Images were captured using the Leica STELLARIS 5 confocal system using a high-resolution oil-immersion objective with a z-step size of 0.25 micrometers. Image stacks were imported into Fiji software⁶² or cochlear length and frequency analysis using the hair cell analysis toolbox (63). The image stacks were imported into IMARIS image-processing software (Oxford Instruments, UK). Base, middle, and apex regions of the cochleae were defined as 10-20%, 30-40%, and 60-70% of the total cochlea length respectively. These locations were chosen to approximate the location of the frequencies 8, 16, and 32 kHz respectively (60). Within each cochlear region, a representative sample of approximately 20 inner hair cells (IHCs) in length was analyzed to measure the length of the sample and count the number of IHCs, outer hair cells (OHCs), and IHC ribbon synapses. The length analyses were done using the measurement system in IMARIS image analysis software on maximum intensity projections of the three-dimensional confocal image stacks. The length was defined as the arc distance of the IHCs juxtaposed to the tectorial membrane. IHCs and OHCs were counted using nuclear staining with CtBP2 within Myo7a expressing cells. DAPI nuclear counterstaining was used to confirm cell counts. The synapses were defined as the colocalization of CtBP2 and PSD95 with opposing voxels within 0.5 micrometers of each other.

### Statistical Analyses

For clinical data, odds-ratios with 95% confidence intervals (OR, 95% confidence interval) and associated p-values were calculated for primary outcomes. Chi-square tests were used to assess baseline covariate proportions before and after PSM. For animal data, male and female mice are presented in aggregate, with separate subgroup analysis separated by sex described in the text. All group comparisons were made using non-parametric student's t test. Comparisons of hair cell counts between cochlear regions were performed using two-way ANOVA with Tukey's ad hoc analysis. Statistical analyses were performed using the TriNetX and the GraphPad-Prism platforms (GraphPad Software Inc., San Diego, CA, USA).

### Results

### Human Cohort Demographics

Demographics for cohorts are detailed in Table 3. From a base population of approximately 130 million patients, 19.5 million carried a diagnosis of hypertension at the time of the query. Patients treated with ARBs or CCBs were compared to those treated with thiazides (control cohort). Cohort comparisons after propensity score matching (PSM), which was done to account for confounding variables, revealed comparable mean age-at-index, sex, and race between ARB-thiazide cohorts (see Table 3) and CCB-thiazide cohorts (see Table 4). Despite the small, clinically insignificant percentage differences between groups after matching, some variables still had p-values <0.05 due to the very large cohort sizes. Chi-square analysis for all demographic and medical covariates for which PSM was applied yielded cohorts with less-than 1% difference in cohort percentages and increases in the p-values across all variables, indicating more group similarity after matching, as expected.

TABLE 3
Characteristics for angiotensin receptor blockade (ARBs) and
thiazide cohorts before and after PSM.

| | | | | ARBs- | Thiazides- | |
|---|---|---|---|---|---|---|
| | ARBs | Thiazides | | PSM | PSM | |
| Variable | (987,977) | (583,261) | p-value | (583,261) | (583,261) | p-value |
| Age-at-Index | 64.0 | 60.0 | <0.0001 | 62.1 | 61.8 | <0.0001 |
| (SD), y | (14.1) | (15.2) | | (14.5) | (14.6) | |
| Female sex, n | 474,445 | 424,003 | <0.0001 | 349,926 | 349,072 | 0.12 |
| (%) | (48.15) | (63.70) | | (60.00) | (59.85) | |
| Male sex, n (%) | 471,509 | 224,654 | <0.0001 | 216,356 | 217,224 | 0.10 |
| | (47.85) | (33.75) | | (37.09) | (37.24) | |
| White, n (%) | 652,046 | 424,698 | <0.0001 | 398,474 | 399,386. | 0.07 |
| | (66.2) | (63.80) | | (68.32) | (6848) | |
| Black or African | 85,330 | 122,217 | <0.0001 | 72,175 | 67,737 | <0.0001 |
| American, n (%) | (8.66) | (18.36) | | (12.37) | (11.61) | |
| Asian, n (%) | 54,341 | 13,035 | <0.0001 | 12,946 | 13,035 | 0.58 |
| | (5.12 | (1.96) | | (2.22) | (2.24) | |
| Diabetes | 212,621 | 73,563 | <0.0001 | 74,414 | 72,587 | <0.0001 |
| Mellitus, n (%) | (21.58) | (11.05) | | (12.76) | (12.45) | |
| Disorders of | 366,778 | 189,673 | <0.0001 | 178,846 | 177,843 | 0.044 |
| lipoprotein | (37.22) | (28.50) | | (30.66) | (30.49) | |
| metabolism and | | | | | | |
| other | | | | | | |
| lipidemias, n | | | | | | |
| (%) | | | | | | |
| Nicotine | 74,193 | 55,328 | <0.0001 | 43,804 | 44,681 | 0.0022 |
| Dependence, n | (7.53) | (8.31) | | (7.51) | (7.66) | |
| (%) | | | | | | |
| Ischemic Heart | 177,483 | 54,226 | <0.0001 | 54,527 | 53,848 | 0.030 |
| Disease | (18.01) | (8.15) | | (9.35) | (9.23) | |
| Cerebrovascular | 77,640 | 35,298 | <0.0001 | 34,130 | 33.551 | 0.022 |
| Diseases n (%) | (7.88) | (5.30) | | (5.85) | (5.75) | |
| Chronic Lower | 134,821 | 87,278 | <0.0001 | 75,554 | 74,978 | 0.11 |
| Respiratory | (13.68) | (13.11) | | (12.95) | (12.86) | |
| Diseases | | | | | | |

TABLE 4
Characteristics for calcium channel blockers (CCBs) and thiazide cohorts
before and after propensity score matching (PSM).

| | | | | Thiazides- | CCBs- | |
|---|---|---|---|---|---|---|
| | Thiazides | CCBs | | PSM | PSM | |
| Variable | (987,977) | (583,261) | p-value | (583,261) | (583,261) | p-value |
| Age-at-Index | 60.0 | 61.4 | <0.0001 | 60.0 | 60.0 | 0.20 |
| (SD), y | (15.2) | (17.2) | | (15.2) | (15.9) | |
| Female sex, n | 424,003 | 636,934 | <0.0001 | 414,492 | 413,839 | 0.24 |
| (%) | (63.70) | (50.46) | | (63.17) | (63.07) | |
| Male sex, n (%) | 224,654 | 581,639 | <0.0001 | 224,654 | 225,210 | 0.31 |
| | (33.75) | (46.08) | | (34.24) | (34.32) | |
| White, n (%) | 424,698 | 683,627 | <0.0001 | 415,187 | 414,556 | 0.25 |
| | (63.80) | (54.16) | | (63.28) | (63.18) | |
| Black or African | 122,217 | 266,634 | <0.0001 | 122,217 | 122,339 | 0.78 |
| American, n (%) | (18.36) | (21.12) | | (18.63) | (18.65) | |
| Asian, n (%) | 13,035 | 62,551 | <0.0001 | 13,035 | 13,080 | 0.78 |
| | (1.96) | (4.96) | | (1.99) | (1.99) | |
| Diabetes | 73,563 | 195,444 | <0.0001 | 73,522 | 72,791 | 0.043 |
| Mellitus, n (%) | (11.05) | (15.48) | | (11.21) | (11.09) | |
| Disorders of | 189,673 | 376,426 | <0.0001 | 186,555 | 188,984 | <0.0001 |
| lipoprotein | (28.50) | (29.82) | | (28.43) | (28.80) | |
| metabolism and | | | | | | |
| other | | | | | | |
| lipidemias, n | | | | | | |
| (%) | | | | | | |
| Nicotine | 55,328 | 134,822 | <0.0001 | 55,259 | 55,062 | 0.54 |
| Dependence, n | (8.31) | (10.68) | | (8.42) | (8.39) | |
| (%) | | | | | | |
| Ischemic Heart | 54,226 | 174,825 | <0.0001 | 54,226 | 54,260 | 0.91 |
| Disease | (8.15) | (13.85) | | (8.27) | (8.27) | |
| Cerebrovascular | 35,298 | 124,969 | <0.0001 | 35,298 | 34,974 | 0.21 |
| Diseases n (%) | (5.30) | (9.90) | | (5.38) | (5.33) | |
| Chronic Lower | 87,278 | 177,077 | <0.0001 | 86,159 | 87,110 | 0.014 |
| Respiratory | (13.11) | (14.03) | | (13.13) | (13.28) | |
| Diseases | | | | | | |

### Protective Associations of First-Line Anti-Hypertensive Agents

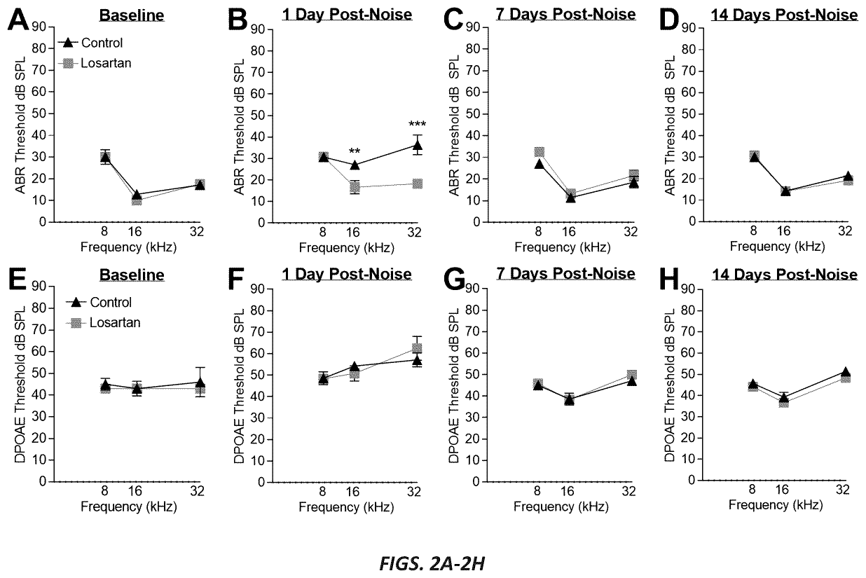

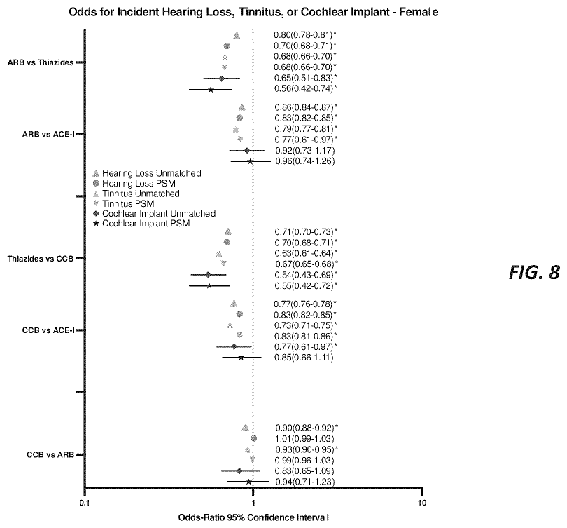

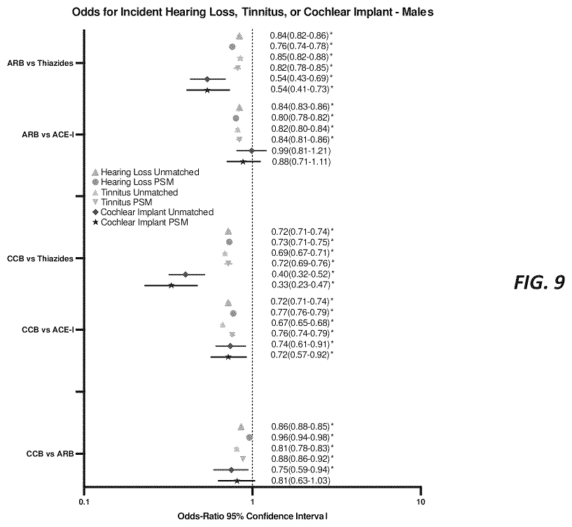

ARBs demonstrate a protective association for hearing-related outcome measures, namely diagnosis of SNHL, tinnitus, or cochlear implantation, before and after PSM when compared to thiazides (FIG. 2). The strongest association in this comparison after PSM was for decreased incidence of cochlear implantation in patients treated with ARBs with OR of 0.56 (95% CI: 0.46-0.69, p<0.0001). For patients treated with ARBs, there was also a substantial decrease in odds of SNHL diagnosis as compared to thiazides with OR of 0.73 (95% CI: 0.72-0.74, p<0.0001). When compared to ACE-I's, ARBs revealed a less pronounced association with improved hearing-related outcomes, but this did not reach statistical significance after group matching with OR of 0.91 (95% CI: 0.76-1.08, p=0.26). When compared directly to CCBs, ARBs demonstrated near equivocal results, with CCBs having a slightly more favorable association with all hearing-related outcome measures with OR of 0.90 (95% CI: 0.75-1.08, p=0.25). CCBs versus thiazides demonstrated an otoprotective effect for CCBs similar to ARBs with the strongest association for cochlear implantation with OR of 0.54 (95% CI: 0.44-0.66, p<0.0001), and for an association with hearing loss, an OR of 0.71 (95% CI: 0.70-0.72, p<0.0001). These comparisons were repeated after stratifying by sex, which demonstrated qualitatively, and quantitatively similar results compared to the pooled data (FIGS. 8 and 9).

### Protection Against Noise-Induced Hearing Loss (NIHL) by Losartan in Mice

To assess the pharmacokinetics of losartan delivered via drug-infused chow in mice, we performed liquid chromatography-tandem mass spectrometric analysis of serum samples to obtain a highly specific and sensitive quantification of levels of losartan and its pharmacologically active and longer-lasting metabolite losartan carboxylic acid (los-COOH), alongside phenacetin, an internal standard introduced as part of the analysis. Losartan was detected at meaningful levels only in serum from treated mice (FIG. 1A). The relatively higher levels of los-COOH levels observed among treated mice (FIG. 1B) is consistent with the rapid metabolism of losartan via cytochrome P450 enzymes and the longer half-life of los-COOH (6-9 hours). As expected, levels of the internal standard were similar between groups demonstrating reliable measurement of another small molecule pharmaceutical using this method (FIG. 1C).

In the first cohort, noise-induced hearing loss was studied using a mild acoustic trauma that induced a temporary threshold shift (TTS) (FIG. 2). In this cohort, mice were subjected to two hours of 8-16k octave band noise exposure at 102.5 dB (males) or 105 dB (females) (64). This resulted in thresholds shifts in auditory brainstem response (ABR) recordings of sound pressure levels (SPLs) at 24 hours after acoustic trauma (FIG. 2A, B), which was significantly blunted by treatment with losartan in response to stimuli at 16 kHz (16 dB vs 5 dB SPL shift, p<0.02) and 32 kHz (18 dB vs. 1 dB SPL shift, p<0.002). Importantly, this treatment effect was also seen in subgroup analysis by sex among males (p=0.02) and to a slightly lesser extent in females (p=0.10). These ABR thresholds shifts were found to be temporary, as thresholds had returned to baseline levels by 7 and 14 days in both groups (FIG. 2C, D). There was no change in DPOAE thresholds after noise exposure and no significant effect of losartan treatment on DPOAE thresholds at any timepoint (FIG. 2 E-H) indicating an absence of significant direct effect of treatment on outer hair cell function. In studies of noise-induced hearing loss wherein temporary shifts have been observed to recovery to baseline thresholds, persistent differences in ABR wave 1 characteristics have been shown to correlate with underlying cochlear dysfunction at the ribbon synapses (23). The ABR wave 1 amplitude growth and wave 1 latencies were calculated to compare this electrophysiologic measure between groups. At 14 days after mild noise-exposure, despite having returned to baseline levels in both groups, losartan treatment resulted in a small but significant effect at preserving ABR wave 1 latencies at the lowest stimulation intensities, suggesting a role in preserving ribbon synapse function.

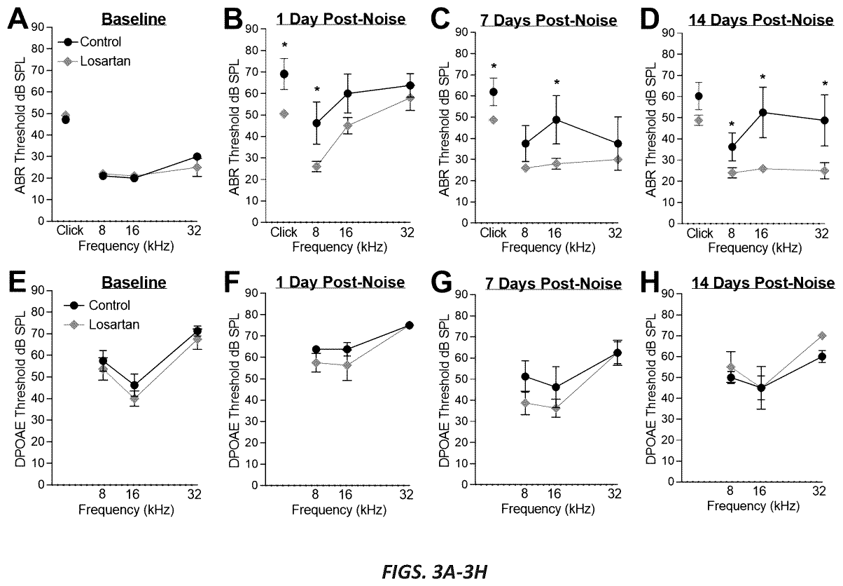

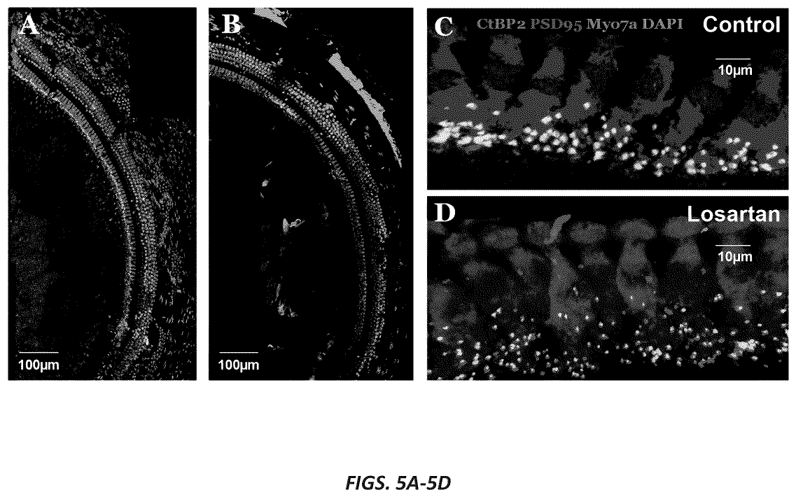

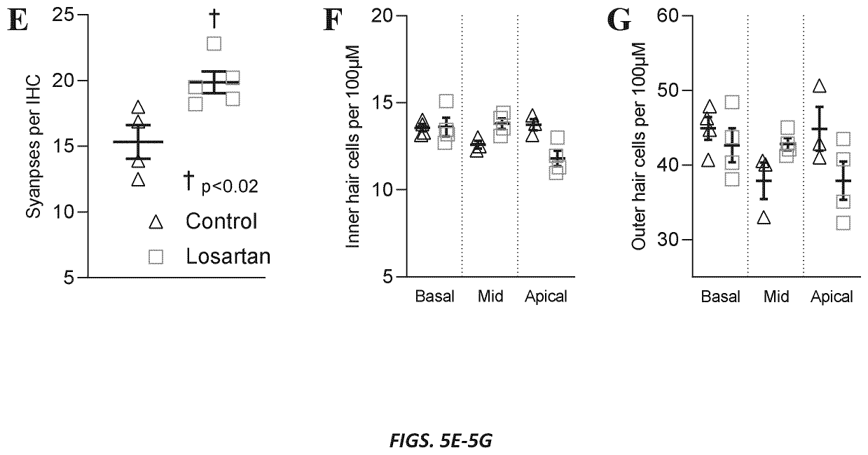

To more directly assess the impact of losartan treatment at preventing synaptopathy at the IHC-ribbon synapses, we dissected cochlear tissues from this lower intensity exposure TTS group for whole mounting, immunostaining, and immunofluorescence image analysis (FIG. 5). Representative cochlear whole mount images (FIG. 5A, B) revealed a normal gross appearance of the inner and outer hair cells rows within the supporting network of the organ of Corti. Staining for ribbon synapses between IHCs and spiral ganglion neurons (SGNs) was performed using antibodies against C-terminal binding protein 2 (CtBP2, green), a pre-synaptic element of the IHCs, and post-synaptic density protein 95 (PSD-95, red), a protein within neurons that anchors neurotransmitter receptors within the synaptic ribbon (FIG. 5, &D). At 14 days after mild noise trauma, the number of synapses per IHC, counted as areas of colocalization of these proteins within 0.5 micrometers of the opposing voxel per hair cell, were found to be significantly higher in the presence of losartan treatment (19.9±0.8 synapses/IHC) as compared to control treatment (15.3±1.3 synapses/IHC, p<0.02) (FIG. 5E). IHC and OHC counts were found to be similar between treatment groups at segments correlating approximately to 8, 16-, and 32-kHz frequencies (p<0.05) (FIG. 5F, G), which is further evidenced by lack of DPOAE threshold changes between treatment groups (FIG. 3E-F). For both groups, there was a significant IHC density difference between cochlear regions with the apical density being less than both the middle and basal turns (p<0.01). There was no difference in OHC density between treatment groups or by cochlear region.

Having identified protection against noise induced temporary ABR threshold shifts, an experiment to test losartan's effect against a more noxious acoustic trauma was performed. In this second cohort, mice were subjected to a moderate intensity noise exposure at 105 dB (males) or 107.5 dB (females) to induce permanent threshold shifts (PTS) (FIG. 3). ABR threshold was additionally assessed in response to a broad-band rapid click stimulation. At 24 hours post-noise exposure, while both groups experienced large ABR thresholds shifts (FIGS. 3A, B), the losartan treated group exhibited significantly (p<0.05) smaller ABR threshold shifts in response to stimuli at 8 kHz (26.5 dB vs. 5.63 dB SPL shift) and 16 kHz (42.12 dB vs. 24.37 dB SPL shift). The protective effect of losartan against ABR threshold increases persisted to 7 days at 16 kHz (26.68 dB vs 8.12 db SPL shift) and click stimuli (17.0 dB vs 4.0 dB SPL shift) and 14 days at 16 kHz (31.1 dB vs 2.5 dB SPL shift) and 32 kHz (18.1 dB vs 1.9 dB SPL shift) (FIG. 3C, D). Again, there was no change in DPOAE thresholds after noise exposure and no significant effect of losartan treatment on DPOAE thresholds at any timepoint (FIG. 3 E-H). Importantly, subgroup analysis by sex revealed significant protection of similar magnitudes against shifts in ABR thresholds at 14 days after noise exposure in both male mice at 32 kHz (p<0.03) and female mice at 16 kHz (p<0.03), analyzed separately (Table 5).

TABLE 5
Unmatched and PSM-adjusted outcomes for each medication comparison. PSM =
propensity score matching. ARB = angiotensin receptor blocker. ACE-I = angiotensin
converting enzyme inhibitor. CCB = calcium channel blocker.

| | | | |
|---|---|---|---|
| | n (%) patients who | Odds-ratio (95% | |
| Cohort (n) | developed outcome | confidence interval) | p-value |

Odds for hearing loss

| | | | |
|---|---|---|---|
| ARB (931,781) | 43,174 (4.63) | 0.83 (0.81-0.84) | <0.0001 |
| Thiazides (629,511) | 629,511 (5.56) | NA | NA |
| ARB-PSM (551,138) | 23,982 (4.32) | 0.73 (0.72-0.74) | <0.0001 |
| Thiazides-PSM | 32,045 (5.83) | NA | NA |
| (549,619) | | | |
| ARB (931,781) | 43,174 (4.63) | 0.86 (0.85-0.87) | <0.0001 |
| ACE-I (1,699,935) | 91,295 (5.37) | NA | NA |
| ARB-PSM (918,811) | 42,532 (4.63) | 0.82 (0.81-0.83) | <0.0001 |
| ACE-I-PSM | 51,533 (5.61) | NA | NA |
| (919,306) | | | |
| ARB (931,781) | 43,174 (4.63) | 1.14 (1.13-1.16) | <0.0001 |
| CCB (1,195,392) | 48,870 (4.09) | NA | NA |
| ARB-PSM (893,060) | 40,562 (4.54) | 1.01 (0.99-1.02) | 0.28 |
| CCB-PSM (888,908) | 40,074 (4.51) | NA | NA |
| Thiazides (629,511) | 35,007 (5.56) | 1.04 (1.02-1.05) | <0.0001 |
| ACE-I (1,699,935) | 91,295 (5.37) | NA | NA |
| Thiazides-PSM | 33,914 (5.66) | 1.13 (1.12-1.15) | <0.0001 |
| (599,010) | | | |
| ACE-I-PSM | 30,415 (5.03) | NA | NA |
| (605,304) | | | |
| Thiazides (629,511) | 35,007 (5.56) | 1.38 (1.36-1.40) | <0.0001 |
| CCB (1,195,392) | 48,870 (4.09) | NA | NA |
| Thiazides-PSM | 34,444 (5.55) | 1.40 (1.38-1.42) | <0.0001 |
| (620,486) | | | |
| CCB-PSM (624,480) | 25,186 (4.03) | NA | NA |
| ACE-I (1,699,935) | 91,295 (5.37) | 1.33 (1.32-1.35) | <0.0001 |
| CCB (1,195,392) | 48,870 (4.09) | NA | NA |
| ACE-I-PSM | 51,374 (5.19) | 1.23 (1.22-1.25) | <0.0001 |
| (989,229) | | | |
| CCB-PSM (985,812) | 41,981 (4.26) | NA | NA |

Odds for tinnitus

| | | | |
|---|---|---|---|
| ARB (964,484) | 17,951 (1.86) | 0.74 (0.72-0.75) | <0.0001 |
| Thiazides (649,559) | 16,273 (2.51) | NA | NA |
| ARB (964,484) | 17,951 (1.86) | 0.82 (0.80-0.83) | <0.0001 |
| ACE-I (1,756,641) | 39,857 (2.27) | NA | NA |
| ARB-PSM (950,822) | 17,764 (1.87) | 0.85 (0.83-0.87) | <0.0001 |
| ACE-I-PSM | 20,896 (2.19) | NA | NA |
| (952,978) | | | |
| ARB (964,484) | 17,951 (1.86) | 1.16 (1.13-1.18) | <0.0001 |
| CCB (1,237,658) | 19,984 (1.62) | NA | NA |
| ARB-PSM (923,670) | 17,029 (1.84) | 1.07 (1.05-1.09) | <0.0001 |
| CCB-PSM (923,897) | 15,968 (1.73) | NA | NA |
| Thiazides (649,559) | 16,273 (2.51) | 1.12 (1.09-1.13) | <0.0001 |
| ACE-I (1,756,641) | 39,857 (2.27) | NA | NA |
| Thiazides-PSM | 15,643 (2.53) | 1.17 (1.15-1.20) | <0.0001 |
| (618,753) | | | |
| ACE-I-PSM | 13,491 (2.17) | NA | NA |
| (623,084) | | | |
| Thiazides (649,559) | 16,273 (2.51) | 1.57 (1.53-1.60) | <0.0001 |
| CCB (1,237,658) | 19,984 (1.62) | NA | NA |
| Thiazides-PSM | 15,982 (2.50) | 1.45 (1.42-1.49) | <0.0001 |
| (640,290) | | | |
| CCB-PSM (643,431) | 11,164 (1.74) | NA | NA |
| ACE-I (1,756,641) | 39,857 (2.27) | 1.42 (1.39-1.44) | <0.0001 |
| CCB (1,237,658) | 19,984 (1.62) | NA | NA |
| ACE-I-PSM | 20,597 (2.01) | 1.21 (1.19-1.23) | <0.0001 |
| (1,025,320) | | | |
| CCB-PSM | 17,055 (1.67) | NA | NA |
| (1,023,034) | | | |

Odds for cochlear implantation

| | | | |
|---|---|---|---|
| ARB (985,120) | 256 (0.026) | 0.62 (0.53-0.74) | <0.0001 |
| Thiazides (665,438) | 277 (0.042) | NA | NA |
| ARB (985,120) | 256 (0.026) | 0.94 (0.81-1.098) | 0.45 |
| ACE-I (1,790,967) | 493 (0.028) | NA | NA |
| ARB-PSM (971,247) | 250 (0.026) | 0.91 (0.76-1.08) | 0.26 |
| ACE-I-PSM | 276 (0.028) | NA | NA |
| (971,244) | | | |
| ARB (985,120) | 256 (0.026) | 1.25 (1.05-1.48) | 0.011 |
| CCB (1,261,966) | 263 (0.021) | NA | NA |
| ARB-PSM (943,374) | 246 (0.026) | 1.11 (0.93-1.134) | 0.25 |
| CCB-PSM (943,339) | 221 (0.023) | NA | NA |
| Thiazides (665,438) | 277 (0.042) | 1.51 (1.31-1.75) | <0.0001 |
| ACE-I (1,790,967) | 493 (0.028) | NA | NA |
| Thiazides-PSM | 272 (0.043) | 1.67 (1.38-2.03) | <0.0001 |
| (634,221) | | | |
| ACE-I-PSM | 163 (0.026) | NA | NA |
| (634,316) | | | |
| Thiazides (665,438) | 277 (0.042) | 2.00 (1.69-2.37) | <0.0001 |
| CCB (1,261,966) | 263 (0.021) | NA | NA |
| Thiazides-PSM | 273 (0.042) | 1.86 (1.52-2.27) | <0.0001 |
| (655,931) | | | |
| CCB-PSM (655,982) | 147 (0.022) | NA | NA |
| ACE-I (1,790,967) | 493 (0.028) | 1.32 (1.14-1.53) | 0.0003 |
| CCB (1,261,966) | 263 (0.021) | NA | NA |
| ACE-I-PSM | 277 (0.027) | 1.18 (0.99-1.40) | 0.063 |
| (1,044,460) | | | |
| CCB-PSM | 235 (0.023) | NA | NA |
| (1,044,431) | | | |

ABR thresholds at 14 days after moderate noise exposure grouped by sex

| | Male | | | Female | | |
|---|---|---|---|---|---|---|
| Frequency | Control | Losartan | P-value | Control | Losartan | P-value |
| 8 kHz (dB HL) | 37.5 | 32.0 | 0.346 | 53.8 | 30.0 | 0.054 |
| 16 kHz (dB HL) | 36.7 | 23.0 | 0.136 | 65.0 | 23.3 | 0.024 |
| 32 kHz (dB HL) | 35.8 | 21.0 | 0.028 | 58.8 | 31.7 | 0.104 |
| Click (dB HL) | 50.0 | 46.7 | 0.350 | 67.5 | 50.0 | 0.207 |

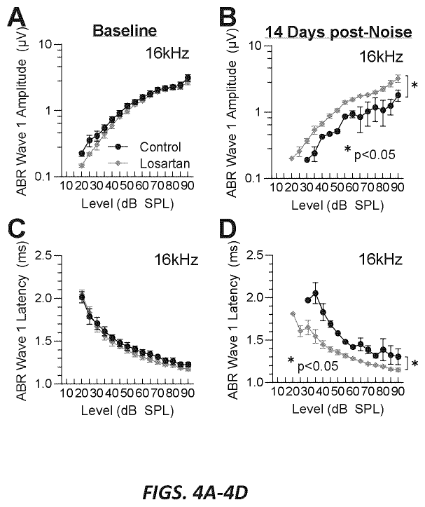

An analysis of ABR wave 1 amplitude and latency among control and losartan-treated animals was performed (FIG. 4). Among mice from the second cohort that had been exposed to higher-intensity noise, losartan significantly preserved both ABR wave 1 amplitude growth function (FIG. 4A, B) and latencies (FIG. 4 C, D) as compared to no treatment at 14 days (p<0.05), with preservation of waveforms with essentially normal baseline characteristics. Given the differences in the ABR thresholds between control and treatment groups at the 14-day timepoint (FIG. 3D), these findings provide confirmatory evidence of the overall impaired auditory function. 10

### DISCUSSION

There are large and unmet medical needs for treatment and prevention of noise-induced hearing loss. Current understanding of the mechanisms of noise-induced hearing loss includes impaired ability in the stria vascularis to maintain the endocochlear potential via blood-labyrinth barrier disruption, excitotoxicity with impaired glutamate recycling, and intertwined acute and chronic cochlear inflammation. Losartan, an angiotensin receptor blocker, is a commonly prescribed FDA-approved medication for hypertension (HTN) and nephroprotection in patients with newly diagnosed type-2 diabetes with a well described safety profile that acts to block inflammatory and vasoconstrictive angiotensin signaling. Our findings that ARBs and CCBs associate with decreased odds for hearing loss in a large human database informed the preclinical studies and further highlight the mechanistic overlap of noise- and HTN-induced hearing loss. In animal experiments, the hypothesis that losartan is a treatment for noise-induced hearing loss in the setting of mild and moderate noise trauma was tested. It was found that treatment with a standard oral dosage of losartan beginning shortly before noise exposure successfully protected against the acute temporary hearing loss after mild noise trauma and further prevented the permanent threshold shifts that persist after moderate noise trauma in mice. Preservation of near-normal ABR thresholds and waveforms with losartan treatment was also found to be associated with preserved ribbon synapse counts.

CCBs have similar biological activity to ARBs with overlapping vasoactive and inflammatory signaling pathways (68). To this end, agents with dual AT1R and calcium channel blockade have been developed (69). L-type calcium channels have been implicated in acoustic injury (70). Lastly, CCBs directly inhibit innate immune activation similar to ARBs (71). While not the focus of these experiments, the incidental association discovered in these experiments with hearing protection for patients with HTN further highlights the mechanistic overlap of noise- and HTN-induced-HL. Indeed, cellular depolarization is an important step in both vasoconstriction and immune cell activation. Thus, blocking depolarization via L-type calcium channels promotes vasodilation and dampens inflammatory cues (72).

The renin angiotensin system (RAS), specifically the action of angiotensin II signaling through its type 1 receptor (AT1R), has been implicated in a multitude of disease states in several organ systems secondary to vasculopathy, inflammatory/oxidative damage, and may play a role in the response to acoustic toxicity. The otoprotection seen in the mouse study secondary to losartan administration may be due to direct downstream effects from antagonism of angiotensin II signaling via the AT1R. It has been found in mice that exposure to acoustic trauma resulting in permanent threshold shifts results from hypoxia followed by decreased cochlear blood flow and vessel caliber of the stria vascularis (25, 65, 66). The mechanism through which this occurs may be secondary to increased genetic expression of pro-vasoconstriction pathways such as endothelin-1 leading to capillary constriction in the spiral ligament and subsequent decreased blood flow (67). It has been well documented that angiotensin II is a potent activator of endothelin-1 production within endothelial cells, mediating subsequent vasoconstriction and vascular remodeling and that inhibition of angiotensin II's action via ARBs attenuates the effects of endothelin-1. As such, it is possible that losartan helps decrease the ischemia associated with noise over-exposure by moderating the vasculature and improving hypoxia.

Losartan is effective in treating vascular and inflammatory diseases in several organ systems, including the auditory system. Ototoxicity secondary to noise has also been attributed to the upregulation of several pro-inflammatory cytokines such as TGF beta, IL6, and TNF alpha and subsequent recruitment of innate immune cells leading to cell death (73-77). Increased expression of adhesion molecules such as ICAM-1 on endothelial cells due to inflammation has been shown to further perpetuate vasculopathy via mechanisms such as neutrophil-mediated vascular injury and generalized immune cell recruitment activity leading to generalized cochleopathy and cochlear cell death (including but not limited to hair cells) (73, 74, 78-80). In cases of hepatic fibrosis, ARB administration significantly decreases TGF-beta expression (81) and inhibits further progression of fibrosis (81, 82). The anti-inflammatory effects of ARBs have also been directly demonstrated within the cochlea as seen in a preclinical model of vestibular schwannoma, where losartan administration resulted in preservation of hearing and normalization of the extracellular matrix contents and vascular distribution likely due to decreased IL-6/STAT3 signaling (51). In a cohort of patients diagnosed with vestibular schwannoma prior to the onset of associated hearing loss, those taking angiotensin receptor blockers were found to have had preserved hearing over several years of follow-up (51), though recent data bring this finding into question (52). In a mouse study examining the effects of hyperglycemic otopathy, losartan was found to protect against hearing loss while preserving normal morphology of the stria vascularis in the treatment group (50). Recently, AT1R blockade using telmisartan was found to protect against aminoglycoside induced hearing loss, with preservation of hair cell counts, possibly through a PPARγ dependent pathway (40, 41), which is part of the innate inflammatory cascade. Similarly, the otoprotection seen in this study may be attributed to dampening of inflammation via losartan administration especially considering that AT1R activation increases production of NF-kB, IL6, and ICAM (45, 46, 83).

The increase in inducible nitric oxide synthase (iNOS) observed in response to cochlear hypoxia and vasoconstriction after acoustic trauma has also been shown to further oxidative damage and hair cell death secondary to excessive production of reactive oxygen species (27, 78, 84). Signaling by angiotensin II through the AT1R is also a significant driver of increased reactive oxygen species generation through activation of NADPH oxidase. Through these multiple mechanisms, the administration of losartan may attenuate cochlear damage by dampening the initial inflammatory and ischemic cascades that drive nitrous oxide overproduction and the associated oxidative stress. In addition, losartan has a tri-cyclic aromatic group similar to other ROS-scavengers, and a fourth aromatic ring capable of accepting free radicals (89-91). Thus, losartan additionally likely exhibits mechanistic overlap with other non-specific ROS-scavengers such as N-acetyl cysteine, D-methionine, and sodium thiosulfate (92-95). Theoretically, this mechanism effects both immune cell-dependent and -independent ROS-related damage. In summary, ARBs may induce their hearing protective effects through several potential mechanisms: preservation of adequate cochlear microcirculation, decreased pro-inflammatory signaling, and decreased oxidative stress via reduced iNOS production and non-specific ROS scavenging from immune cell-dependent and -independent sources. Uncovering the contributions of AT1R blockade in each of these areas will the topic of future mechanistic study.

One additional mechanism by which losartan may provide hearing protection in the face of noise toxicity may be via a positive feedback loop involving aldosterone. Interestingly, there have been several studies documenting the otoprotective effect that aldosterone has on presbycusis. Because one aspect of the pathophysiology of presbycusis has been thought to be loss of ability to generate adequate endocochlear potential, aldosterone's physiologic action of upregulating potassium channels such as NKCC may afford restoration of endocochlear potential and therefore alleviate age related hearing loss. In the case of NIHL where one of the primary insults to tissue is secondary to overexcitation and glutamate toxicity, inhibition of aldosterone through losartan may contribute to potential otoprotection by inhibiting aldosterone-mediated upregulation of ion channels thus decreasing endocochlear potential, dampening noise-induced excitotoxicity, and rescuing synaptopathy.

### BIBLIOGRAPHY

1. Lin F R, Thorpe R, Gordon-Salant S, Ferrucci L. Hearing loss prevalence and risk factors among older adults in the United States. J Gerontol A Biol Sci Med Sci. May 2011; 66 (5): 582-90. doi: 10.1093/gerona/glr002
2. Kim A S, Garcia Morales E E, Amjad H, et al. Association of Hearing Loss With Neuropsychiatric Symptoms in Older Adults With Cognitive Impairment. Am J Geriatr Psychiatry. June 2021; 29 (6): 544-553. doi: 10.1016/j.jagp.2020.10.002
3. Babajanian E E, Patel N S, Gurgel R K. The Impact of Cochlear Implantation: Cognitive Function, Quality of Life, and Frailty in Older Adults. Semin Hear. November 2021; 42 (4): 342-351. doi: 10.1055/s-0041-1739367
4. Cunningham L L, Tucci D L. Hearing Loss in Adults. N Engl J Med. Dec. 21 2017; 377 (25): 2465-2473. doi: 10.1056/NEJMra1616601
5. Y I C, J E, F S. Vital Signs: Noise-Induced Hearing Loss Among Adults MMWR Morb Mortal Wkly Rep 2017. p. 139-144.
6. Liu Y, Chen Q, Xu Y. Research progress in refractory sudden hearing loss: steroid therapy. J Int Med Res. January 2020; 48 (1): 300060519889426. doi: 10.1177/0300060519889426
7. Wei B P, Stathopoulos D, O'Leary S. Steroids for idiopathic sudden sensorineural hearing loss. Cochrane Database Syst Rev. Jul. 2 2013; 2013 (7): Cd003998. doi: 10.1002/14651858.CD003998.pub3 8 Rozbicki P, Usowski J, Siewiera J, Jurkiewicz D. The Influence of Steroid Therapy on the Treatment Results in Patients with Sudden Sensorineural Hearing Loss. J Clin Med. Oct. 15 2022; 11 (20) doi: 10.3390/jcm11206085
9. Gioacchini F M, Pisani D, Viola P, et al. Diabetes Mellitus and Hearing Loss: A Complex Relationship. Medicina (Kaunas). Jan. 30 2023; 59 (2) doi: 10.3390/medicina59020269
10. Hara K, Okada M, Takagi D, et al. Association between hypertension, dyslipidemia, and diabetes and prevalence of hearing impairment in Japan. Hypertens Res. September 2020; 43 (9): 963-968. doi: 10.1038/s41440-020-0444-y
11. Bener A, Al-Hamaq A, Abdulhadi K, Salahaldin A H, Gansan L. Interaction between diabetes mellitus and hypertension on risk of hearing loss in highly endogamous population. Diabetes Metab Syndr. November 2017; 11 Suppl 1: S45-s51. doi: 10.1016/j.dsx.2016.09.004
12. Nawaz M U, Vinayak S, Rivera E, et al. Association Between Hypertension and Hearing Loss. Cureus. September 2021; 13 (9): e18025. doi: 10.7759/cureus.18025
13. Patel S D, Patel S, Finberg A, Shah V N, Mittal R, Eshraghi A A. Association Between Tinnitus and Hypertension: A Cross-Sectional Analysis of the National Health and Nutrition Examination Survey. Otol Neurotol. Aug. 1 2022; 43 (7): 766-772. doi: 10.1097/mao.0000000000003579
14. Ramage-Morin P L, Gilmour H, Banks R, Pineault D, Atrach M. Hypertension associated with hearing health problems among Canadian adults aged 19 to 79 years. Health Rep. Oct. 20 2021; 32 (10): 14-26. doi: 10.25318/82-003-x202101000002-eng
15. Rim H S, Kim M G, Park D C, et al. Association of Metabolic Syndrome with Sensorineural Hearing Loss. J Clin Med. Oct. 22 2021; 10 (21) doi: 10.3390/jcm10214866
16. Xu Y, Wang G, Wang M, et al. Association of metabolic syndrome and its components with hearing loss in low-income women: A population-based cross-sectional study. Clinical Epidemiology and Global Health; 2024.
17. Chang T Y, Liu C S, Huang K H, Chen R Y, Lai J S, Bao B Y. High-frequency hearing loss, occupational noise exposure and hypertension: a cross-sectional study in male workers. Environ Health. Apr. 25 2011; 10:35. doi: 10.1186/1476-069x-10-35
18. Jin N, Zhong H Y, Yuan F, Deng H X. Positive Associations between Bilateral High-Frequency Hearing Loss and Hypertension Risk in Short-Term Occupational Noise Exposure. Biomed Environ Sci. Dec. 20 2021; 34 (12): 992-997. doi: 10.3967/bes2021.134
19. Li W, Yi G, Chen Z, et al. Association of occupational noise exposure, bilateral hearing loss with hypertension among Chinese workers. J Hypertens. Apr. 1 2021; 39 (4): 643-650. doi: 10.1097/hjh.0000000000002696
20. Gates G A, Schmid P, Kujawa S G, Nam B, D'Agostino R. Longitudinal threshold changes in older men with audiometric notches. Hear Res. March 2000; 141 (1-2): 220-8. doi: 10.1016/s0378-5955 (99) 00223-3
21. Kujawa S G, Liberman M C. Acceleration of age-related hearing loss by early noise exposure: evidence of a misspent youth. J Neurosci. Feb. 15 2006; 26 (7): 2115-23. doi: 10.1523/jneurosci.4985-05.2006
22. Kujawa S G, Liberman M C. Synaptopathy in the noise-exposed and aging cochlea: Primary neural degeneration in acquired sensorineural hearing loss. Hear Res. December 2015; 330 (Pt B): 191-9. doi: 10.1016/j.heares.2015.02.009
23. Kujawa S G, Liberman M C. Adding insult to injury: cochlear nerve degeneration after “temporary” noise-induced hearing loss. J Neurosci. Nov. 11 2009; 29 (45): 14077-85. doi: 10.1523/jneurosci.2845-09.2009
24. Liberman M C, Kujawa S G. Cochlear synaptopathy in acquired sensorineural hearing loss: Manifestations and mechanisms. Hear Res. June 2017; 349:138-147. doi: 10.1016/j.heares.2017.01.003
25. Shin S A, Lyu A R, Jeong S H, Kim T H, Park M J, Park Y H. Acoustic Trauma Modulates Cochlear Blood Flow and Vasoactive Factors in a Rodent Model of Noise-Induced Hearing Loss. Int J Mol Sci. Oct. 25 2019; 20 (21) doi: 10.3390/ijms20215316
26. Wu J, Han W, Chen X, et al. Matrix metalloproteinase-2 and -9 contribute to functional integrity and noise-induced damage to the blood-labyrinth-barrier. Mol Med Rep. August 2017; 16 (2): 1731-1738. doi: 10.3892/mmr.2017.6784
27. Shi X, Nuttall A L. Upregulated iNOS and oxidative damage to the cochlear stria vascularis due to noise stress. Brain Res. Mar. 28 2003; 967 (1-2): 1-10. doi: 10.1016/s0006-8993 (02) 04090-8
28. Ma K, Zhang A, She X, et al. Disruption of Glutamate Release and Uptake-Related Protein Expression After Noise-Induced Synaptopathy in the Cochlea. Front Cell Dev Biol. 2021; 9:720902. doi: 10.3389/fcell.2021.720902
29. Hu N, Rutherford M A, Green S H. Protection of cochlear synapses from noise-induced excitotoxic trauma by blockade of Ca(2+)-permeable AMPA receptors. Proc Natl Acad Sci USA. Feb. 18 2020; 117 (7): 3828-3838. doi: 10.1073/pnas. 1914247117
30. Paciello F, Pisani A, Rolesi R, et al. Noise-Induced Cochlear Damage Involves PPAR Down-Regulation through the Interplay between Oxidative Stress and Inflammation. Antioxidants (Basel). Jul. 26 2021; 10 (8) doi: 10.3390/antiox10081188
31. Fujioka M, Kanzaki S, Okano H J, Masuda M, Ogawa K, Okano H. Proinflammatory cytokines expression in noise-induced damaged cochlea. J Neurosci Res. March 2006; 83 (4): 575-83. doi: 10.1002/jnr.20764
32. Wan G, Gómez-Casati M E, Gigliello A R, Liberman M C, Corfas G. Neurotrophin-3 regulates ribbon synapse density in the cochlea and induces synapse regeneration after acoustic trauma. Elife. Oct. 20 2014; 3doi: 10.7554/eLife.03564
33. Cassinotti L R, Ji L, Borges B C, et al. Cochlear Neurotrophin-3 overexpression at mid-life prevents age-related inner hair cell synaptopathy and slows age-related hearing loss. Aging Cell. October 2022; 21 (10): e13708. doi: 10.1111/acel.13708
34. Fetoni A R, Pisani A, Rolesi R, et al. Early Noise-Induced Hearing Loss Accelerates Presbycusis Altering Aging Processes in the Cochlea. Front Aging Neurosci. 2022; 14:803973. doi: 10.3389/fnagi.2022.803973
35. Mezzano S A, Ruiz-Ortega M, Egido J. Angiotensin II and renal fibrosis. Hypertension. September 2001; 38 (3 Pt 2): 635-8. doi: 10.1161/hy09t1.094234
36. Gupta D, Kumar A, Mandloi A, Shenoy V. Renin angiotensin aldosterone system in pulmonary fibrosis: Pathogenesis to therapeutic possibilities. Pharmacol Res. December 2021; 174:105924. doi: 10.1016/j.phrs.2021.105924
37. Wang J, Chen L, Chen B, et al. Chronic Activation of the Renin-Angiotensin System Induces Lung Fibrosis. Sci Rep. Oct. 23 2015; 5:15561. doi: 10.1038/srep15561
38. Bennion D M, Haltigan E, Regenhardt R W, Steckelings U M, Sumners C. Neuroprotective mechanisms of the ACE2-angiotensin-(1-7)-Mas axis in stroke. Curr Hypertens Rep. February 2015; 17 (2): 3. doi: 10.1007/s11906-014-0512-2
39. Abiodun O A, Ola M S. Role of brain renin angiotensin system in neurodegeneration: An update. Saudi J Biol Sci. March 2020; 27 (3): 905-912. doi: 10.1016/j.sjbs.2020.01.026
40. Díez J, Querejeta R, López B, González A, Larman M, Martínez Ubago J L. Losartan-dependent regression of myocardial fibrosis is associated with reduction of left ventricular chamber stiffness in hypertensive patients. Circulation. May 28 2002; 105 (21): 2512-7. doi: 10.1161/01.cir.0000017264.66561.3d
41. Miguel-Carrasco J L, Beaumont J, San José G, et al. Mechanisms underlying the cardiac antifibrotic effects of losartan metabolites. Sci Rep. Feb. 3 2017; 7:41865. doi: 10.1038/srep41865
42. Clarke S L. Hypertension in Adults: Initial Evaluation and Management. Am Fam Physician. October 2023; 108 (4): 352-359.
43. Lindhardt M, Persson F, Currie G, et al. Proteomic prediction and Renin angiotensin aldosterone system Inhibition prevention Of early diabetic nephRopathy in TYpe 2 diabetic patients with normoalbuminuria (PRIORITY): essential study design and rationale of a randomised clinical multicentre trial. BMJ Open. Mar. 2 2016; 6 (3): e010310. doi: 10.1136/bmjopen-2015-010310
44. Border W A, Noble N A. Interactions of transforming growth factor-beta and angiotensin II in renal fibrosis. Hypertension. January 1998; 31 (1 Pt 2): 181-8. doi:10.1161/01.hyp.31.1.181
45. Pacurari M, Kafoury R, Tchounwou P B, Ndebele K. The Renin-Angiotensin-aldosterone system in vascular inflammation and remodeling. Int J Inflam. 2014; 2014:689360. doi: 10.1155/2014/689360
46. Satou R, Penrose H, Navar L G. Inflammation as a Regulator of the Renin-Angiotensin System and Blood Pressure. Curr Hypertens Rep. Oct. 5 2018; 20 (12): 100. doi: 10.1007/s11906-018-0900-0
47. Cortada M, Wei E, Jain N, Levano S, Bodmer D. Telmisartan Protects Auditory Hair Cells from Gentamicin-Induced Toxicity in vitro. Audiol Neurootol. 2020; 25 (6): 297-308. doi: 10.1159/000506796
48. Lee C H, Lee S M, Kim S Y. Telmisartan Attenuates Kanamycin-Induced Ototoxicity in Rats. Int J Mol Sci. Nov. 24 2021; 22 (23) doi: 10.3390/ijms222312716
49. Cosgrove D, Gratton M A, Madison J, et al. Dual inhibition of the endothelin and angiotensin receptor ameliorates renal and inner ear pathologies in Alport mice. J Pathol. July 2023; 260 (3): 353-364. doi: 10.1002/path.6087
50. Meyer zum Gottesberge A M, Massing T, Sasse A, Palma S, Hansen S. Zucker diabetic fatty rats, a model for type 2 diabetes, develop an inner ear dysfunction that can be attenuated by losartan treatment. Cell Tissue Res. November 2015; 362 (2): 307-15. doi: 10.1007/s00441-015-2215-7
51. Wu L, Vasilijic S, Sun Y, et al. Losartan prevents tumor-induced hearing loss and augments radiation efficacy in NF2 schwannoma rodent models. Sci Transl Med. Jul. 14 2021; 13 (602) doi: 10.1126/scitranslmed.abd4816
52. Lovin B D, Nader M E, Qing Y, et al. Losartan May Not Prevent Vestibular Schwannoma Growth or Related Hearing Loss During Observation. Otol Neurotol. Jul. 1 2024; 45 (6): 690-695. doi: 10.1097/mao.0000000000004214
53. Yancey K L, Barnett S L, Kutz W, et al. Hearing Preservation After Intervention in Vestibular Schwannoma. Otol Neurotol. Sep. 1 2022; 43 (8): e846-e855. doi: 10.1097/mao.0000000000003618
54. Franchetti Y. Use of Propensity Scoring and Its Application to Real-World Data: Advantages, Disadvantages, and Methodological Objectives Explained to Researchers Without Using Mathematical Equations. J Clin Pharmacol. March 2022; 62 (3): 304-319. doi: 10.1002/jcph.1989
55. Benedetto U, Head S J, Angelini G D, Blackstone E H. Statistical primer: propensity score matching and its alternatives. Eur J Cardiothorac Surg. Jun. 1 2018; 53 (6): 1112-1117. doi: 10.1093/ejcts/ezyl67
56. Cioci A C, Cioci A L, Mantero AMA, Parreco J P, Yeh D D, Rattan R. Advanced Statistics: Multiple Logistic Regression, Cox Proportional Hazards, and Propensity Scores. Surg Infect (Larchmt). August 2021; 22 (6): 604-610. doi: 10.1089/sur.2020.425
57. Park C R, Willott J F, Walton J P. Age-related changes of auditory sensitivity across the life span of CBA/CaJ mice. Hear Res. January 2024; 441:108921. doi: 10.1016/j.heares.2023.108921
58. Liu H, Peng H, Wang L, Xu P, Wang Z, Wu H. Differences in Calcium Clearance at Inner Hair Cell Active Zones May Underlie the Difference in Susceptibility to Noise-Induced Cochlea Synaptopathy of C57B L/6J and CBA/CaJ Mice. Front Cell Dev Biol. 2020; 8:635201. doi: 10.3389/fcell.2020.635201
59. Puris E, Pasanen M, Gynther M, et al. A liquid chromatography-tandem mass spectrometry analysis of nine cytochrome P450 probe drugs and their corresponding metabolites in human serum and urine. Anal Bioanal Chem. January 2017; 409 (1): 251-268. doi: 10.1007/s00216-016-9994-x
60 Iwasa Y, Klimara M J, Yoshimura H, et al. Mutation-agnostic RNA interference with engineered replacement rescues Tmc1-related hearing loss. Life Sci Alliance. March 2023; 6 (3) doi: 10.26508/1sa.202201592
61. Fang Q J, Wu F, Chai R, Sha S H. Cochlear Surface Preparation in the Adult Mouse. J Vis Exp. Nov. 6 2019; (153) doi: 10.3791/60299
62. Schindelin J, Arganda-Carreras I, Frise E, et al. Fiji: an open-source platform for biological-image analysis. Nat Methods. Jun. 28 2012; 9 (7): 676-82. doi:10.1038/nmeth.2019
63. Buswinka C J, Osgood R T, Simikyan R G, Rosenberg D B, Indzhykulian A A. The hair cell analysis toolbox is a precise and fully automated pipeline for whole cochlea hair cell quantification. PLOS Biol. March 2023; 21 (3): e3002041. doi: 10.1371/journal.pbio.3002041
64. Fernandez K A, Guo D, Micucci S, De Gruttola V, Liberman M C, Kujawa S G. Noise-induced Cochlear Synaptopathy with and Without Sensory Cell Loss. Neuroscience. Feb. 10 2020; 427:43-57. doi: 10.1016/j.neuroscience.2019.11.051
65. Lamm K, Arnold W. Noise-induced cochlear hypoxia is intensity dependent, correlates with hearing loss and precedes reduction of cochlear blood flow. Audiol Neurootol. May-June 1996; 1 (3): 148-60. doi: 10.1159/000259195
66. Axelsson A, Dengerink H. The effects of noise on histological measures of the cochlear vasculature and red blood cells: a review. Hear Res. December 1987; 31 (2): 183-91. doi: 10.1016/0378-5955 (87) 90125-0
67. Sadanaga M, Liu J, Wangemann P. Endothelin-A receptors mediate vasoconstriction of capillaries in the spiral ligament. Hear Res. October 1997; 112 (1-2): 106-14. doi: 10.1016/s0378-5955 (97) 00121-4
68. Toba H, Nakagawa Y, Miki S, et al. Calcium channel blockades exhibit anti-inflammatory and antioxidative effects by augmentation of endothelial nitric oxide synthase and the inhibition of angiotensin converting enzyme in the N (G)-nitro-L-arginine methyl ester-induced hypertensive rat aorta: vasoprotective effects beyond the blood pressure-lowering effects of amlodipine and manidipine. Hypertens Res. August 2005; 28 (8): 689-700. doi: 10.1291/hypres.28.689
69. Mahgoub S, Kotb El-Sayed M I, El-Shehry M F, Mohamed Awad S, Mansour Y E, Fatahala S S. Synthesis of novel calcium channel blockers with ACE2 inhibition and dual antihypertensive/anti-inflammatory effects: A possible therapeutic tool for COVID-19. Bioorg Chem. November 2021; 116:105272. doi: 10.1016/j.bioorg.2021.105272
70. Uemaetomari I, Tabuchi K, Nakamagoe M, Tanaka S, Murashita H, Hara A. L-type voltage-gated calcium channel is involved in the pathogenesis of acoustic injury in the cochlea. Tohoku J Exp Med. May 2009; 218 (1): 41-7. doi: 10.1620/tjem.218.41
71. Liu W, Matsumori A. Calcium channel blockers and modulation of innate immunity. Curr Opin Infect Dis. June 2011; 24 (3): 254-8. doi: 10.1097/QCO.0b013e3283463e5b
72. Rosales C, Brown E J. Calcium channel blockers nifedipine and diltiazem inhibit Ca2+ release from intracellular stores in neutrophils. J Biol Chem. Jan. 25 1992; 267 (3): 1443-8.
73. Tan W J, Thorne P R, Vlajkovic S M. Characterisation of cochlear inflammation in mice following acute and chronic noise exposure. Histochem Cell Biol. August 2016; 146 (2): 219-30. doi: 10.1007/s00418-016-1436-5
74. Infante E B, Channer G A, Telischi F F, et al. Mannitol protects hair cells against tumor necrosis factor α-induced loss. Otol Neurotol. December 2012; 33 (9): 1656-63. doi: 10.1097/MAO.0b013e31826bedd9
75. Rai V, Wood M B, Feng H, Schabla N M, Tu S, Zuo J. The immune response after noise damage in the cochlea is characterized by a heterogeneous mix of adaptive and innate immune cells. Sci Rep. Sep. 16 2020; 10 (1): 15167. doi: 10.1038/s41598-020-72181-6
76. Zhang C, Frye M D, Riordan J, et al. Loss of CX3CR1 augments neutrophil infiltration into cochlear tissues after acoustic overstimulation. J Neurosci Res. November 2021; 99 (11): 2999-3020. doi: 10.1002/jnr.24925
77. Li P, Qian T, Sun S. Spatial architecture of the cochlear immune microenvironment in noise-induced and age-related sensorineural hearing loss. Int Immunopharmacol. January 2023; 114:109488. doi: 10.1016/j.intimp.2022.109488
78. Park J S, Jou I, Park S M. Attenuation of noise-induced hearing loss using methylene blue. Cell Death Dis. Apr. 24 2014; 5 (4): e1200. doi: 10.1038/cddis.2014.170
79. Urdang Z D, Bills J L, Cahana D Y, Muldoon L L, Neuwelt E A. Toll-like Receptor 4 Signaling and Downstream Neutrophilic Inflammation Mediate Endotoxemia-Enhanced Blood-Labyrinth Barrier Trafficking. Otol Neurotol. January 2020; 41 (1): 123-132. doi: 10.1097/mao.0000000000002447
80. Harlan J M. Neutrophil-mediated vascular injury. Acta Med Scand Suppl. 1987; 715:123-9. doi: 10.1111/j.0954-6820.1987.tb09912.x
81. Yoshiji H, Kuriyama S, Yoshii J, et al. Angiotensin-II type 1 receptor interaction is a major regulator for liver fibrosis development in rats. Hepatology. October 2001; 34 (4 Pt 1): 745-50. doi: 10.1053/jhep.2001.28231
82. Salama Z A, Sadek A, Abdelhady A M, Darweesh S K, Morsy S A, Esmat G. Losartan may inhibit the progression of liver fibrosis in chronic HCV patients. Hepatobiliary Surg Nutr. June 2016; 5 (3): 249-55. doi: 10.21037/hbsn.2016.02.06
83. Hoogwerf B J. Renin-angiotensin system blockade and cardiovascular and renal protection. Am J Cardiol. Jan. 4 2010; 105 (1 Suppl): 30a-5a. doi: 10.1016/j.amjcard.2009.10.009
84. Yamane H, Takayama M, Sunami K, et al. Nitric oxide induces apoptosis of the hair cells of cochlea. Acta Otolaryngol Suppl. October 2004; (554): 6-11. doi: 10.1080/03655230410018453
85. Millatt L J, Abdel-Rahman E M, Siragy H M. Angiotensin II and nitric oxide: a question of balance. Regul Pept. May 31 1999; 81 (1-3): 1-10. doi: 10.1016/s0167-0115 (99) 00027-0
86. Ichiki T, Usui M, Kato M, et al. Downregulation of angiotensin II type 1 receptor gene transcription by nitric oxide. Hypertension. January 1998; 31 (1 Pt 2): 342-8. doi: 10.1161/01.hyp.31.1.342
87. Suschek C V, Schnorr O, Kolb-Bachofen V. The role of iNOS in chronic inflammatory processes in vivo: is it damage-promoting, protective, or active at all? Curr Mol Med. November 2004; 4 (7): 763-75. doi: 10.2174/1566524043359908
88. Nakazawa H, Chang K, Shinozaki S, et al. iNOS as a Driver of Inflammation and Apoptosis in Mouse Skeletal Muscle after Burn Injury: Possible Involvement of Sirt1 S-Nitrosylation-Mediated Acetylation of p65 NF-κB and p53. PLOS One. 2017; 12 (1): e0170391. doi: 10.1371/journal.pone.0170391
89. Shao J, Nangaku M, Inagi R, et al. Receptor-independent intracellular radical scavenging activity of an angiotensin II receptor blocker. J Hypertens. August 2007; 25 (8): 1643-9. doi: 10.1097/HJH.0b013e328165d159
90. Ge L, Zhang G, You B, Cheng G, Chen L, Shi R. The role of losartan in preventing vascular remodeling in spontaneously hypertensive rats by inhibition of the H(2)O(2)/VPO1/HOCl/MMPs pathway. Biochem Biophys Res Commun. Nov. 4 2017; 493 (1): 855-861. doi: 10.1016/j.bbrc.2017.06.026
91. Yao E H, Fukuda N, Matsumoto T, et al. Losartan improves the impaired function of endothelial progenitor cells in hypertension via an antioxidant effect. Hypertens Res. November 2007; 30 (11): 1119-28. doi: 10.1291/hypres.30.1119
92. Alagic Z, Goiny M, Canlon B. Protection against acoustic trauma by direct application of D-methionine to the inner ear. Acta Otolaryngol. August 2011; 131 (8): 802-8. doi: 10.3109/00016489.2011.564652
93. Campbell K, Claussen A, Meech R, Verhulst S, Fox D, Hughes L. D-methionine (D-met) significantly rescues noise-induced hearing loss: timing studies. Hear Res. December 2011; 282 (1-2): 138-44. doi: 10.1016/j.heares.2011.08.003
94. Fetoni A R, Ralli M, Sergi B, Parrilla C, Troiani D, Paludetti G. Protective effects of N-acetylcysteine on noise-induced hearing loss in guinea pigs. Acta Otorhinolaryngol Ital. April 2009; 29 (2): 70-5.
95. Crowson M G, Hertzano R, Tucci D L. Emerging Therapies for Sensorineural Hearing Loss. Otol Neurotol. July 2017; 38 (6): 792-803. doi: 10.1097/mao.0000000000001427
96. Jeong M, Ocwieja K E, Han D, et al. Direct SARS-COV-2 infection of the human inner ear may underlie COVID-19-associated audiovestibular dysfunction. Commun Med (Lond). 2021; 1 (1): 44. doi: 10.1038/s43856-021-00044-w
97. Villela D, Leonhardt J, Patel N, et al. Angiotensin type 2 receptor (AT2R) and receptor Mas: a complex liaison. Clin Sci (Lond). February 2015; 128 (4): 227-34. doi: 10.1042/cs20130515
98. Liu H, Chen L, Giffen K P, et al. Cell-Specific Transcriptome Analysis Shows That Adult Pillar and Deiters' Cells Express Genes Encoding Machinery for Specializations of Cochlear Hair Cells. Front Mol Neurosci. 2018; 11:356. doi: 10.3389/fnmol.2018.00356
99. Kolla L, Kelly M C, Mann Z F, et al. Characterization of the development of the mouse cochlear epithelium at the single cell level. Nat Commun. May 13 2020; 11 (1): 2389. doi: 10.1038/s41467-020-16113-y
100. Cederroth C R, Park J S, Basinou V, et al. Circadian Regulation of Cochlear Sensitivity to Noise by Circulating Glucocorticoids. Curr Biol. Aug. 5 2019; 29 (15): 2477-2487.e6. doi: 10.1016/j.cub.2019.06.057
101. de Kloet A D, Steckelings U M, Sumners C. Protective Angiotensin Type 2 Receptors in the Brain and Hypertension. Curr Hypertens Rep. June 2017; 19 (6): 46. doi: 10.1007/s11906-017-0746-x
102. Matavelli L C, Siragy H M. AT2 receptor activities and pathophysiological implications. J Cardiovasc Pharmacol. March 2015; 65 (3): 226-32. doi: 10.1097/fjc.0000000000000208
103. Xu P, Sriramula S, Lazartigues E. ACE2/ANG-(1-7)/Mas pathway in the brain: the axis of good. Am J Physiol Regul Integr Comp Physiol. April 2011; 300 (4): R804-17. doi: 10.1152/ajpregu.00222.2010
104. Su Z, Zimpelmann J, Burns K D. Angiotensin-(1-7) inhibits angiotensin II-stimulated phosphorylation of MAP kinases in proximal tubular cells. Kidney Int. June 2006; 69 (12): 2212-8. doi: 10.1038/sj.ki.5001509
105. Moon J Y, Tanimoto M, Gohda T, et al. Attenuating effect of angiotensin-(1-7) on angiotensin II-mediated NAD (P) H oxidase activation in type 2 diabetic nephropathy of K K-A (y)/Ta mice. Am J Physiol Renal Physiol. June 2011; 300 (6): F1271-82. doi: 10.1152/ajprenal.00065.2010
106. Farooqui Z, Banday A A. Angiotensin 1-7 exerts antioxidant effects, suppresses Mammalian Target of Rapamycin (mTOR) signaling, and inhibits apoptosis in renal proximal tubular cells. Peptides. February 2024; 172:171136. doi: 10.1016/j.peptides.2023.171136
107. Grobe J L, Mecca A P, Lingis M, et al. Prevention of angiotensin I I-induced cardiac remodeling by angiotensin-(1-7). Am J Physiol Heart Circ Physiol. February 2007; 292 (2): H736-42. doi: 10.1152/ajpheart.00937.2006
108. Shah A, Oh Y B, Lee S H, Lim J M, Kim S H. Angiotensin-(1-7) attenuates hypertension in exercise-trained renal hypertensive rats. Am J Physiol Heart Circ Physiol. Jun. 1 2012; 302 (11): H2372-80. doi: 10.1152/ajpheart.00846.2011
109. Shenoy V, Ferreira A J, Qi Y, et al. The angiotensin-converting enzyme 2/angiogenesis-(1-7)/Mas axis confers cardiopulmonary protection against lung fibrosis and pulmonary hypertension. Am J Respir Crit Care Med. Oct. 15 2010; 182 (8): 1065-72. doi: 10.1164/rccm.200912-18400C
110. Pereira R M, Dos Santos R A, Teixeira M M, et al. The renin-angiotensin system in a rat model of hepatic fibrosis: evidence for a protective role of Angiotensin-(1-7). J Hepatol. April 2007; 46 (4): 674-81. doi: 10.1016/j.jhep.2006.10.018
111. Yamamoto K, Ohishi M, Katsuya T, et al. Deletion of angiotensin-converting enzyme 2 accelerates pressure overload-induced cardiac dysfunction by increasing local angiotensin II. Hypertension. April 2006; 47 (4): 718-26. doi: 10.1161/01.HYP.0000205833.89478.5b
112. Gava E, de Castro C H, Ferreira A J, et al. Angiotensin-(1-7) receptor Mas is an essential modulator of extracellular matrix protein expression in the heart. Regul Pept. Apr. 10 2012; 175 (1-3): 30-42. doi: 10.1016/j.regpep.2012.01.001
113. Sukumaran V, Veeraveedu P T, Gurusamy N, et al. Telmisartan acts through the modulation of ACE-2/ANG 1-7/mas receptor in rats with dilated cardiomyopathy induced by experimental autoimmune myocarditis. Life Sci. Feb. 13 2012; 90 (7-8): 289-300. doi: 10.1016/j.lfs.2011.11.018
114. Sukumaran V, Veeraveedu P T, Gurusamy N, et al. Cardioprotective effects of telmisartan against heart failure in rats induced by experimental autoimmune myocarditis through the modulation of angiotensin-converting enzyme-2/angiotensin 1-7/mas receptor axis. Int J Biol Sci. 2011; 7 (8): 1077-92. doi: 10.7150/ijbs.7.1077
115. Benigni A, Cassis P, Remuzzi G. Angiotensin II revisited: new roles in inflammation, immunology and aging. EMBO Mol Med. July 2010; 2 (7): 247-57. doi: 10.1002/emmm.201000080
116. Young M J. Mechanisms of mineralocorticoid receptor-mediated cardiac fibrosis and vascular inflammation. Curr Opin Nephrol Hypertens. March 2008; 17 (2): 174-80. doi: 10.1097/MNH.0b013e3282f56854
117. Verma A, Vaidya A, Subudhi S, Waikar S S. Aldosterone in chronic kidney disease and renal outcomes. Eur Heart J. Oct. 11 2022; 43 (38): 3781-3791. doi: 10.1093/eurheartj/ehac352
118. Toto R D. Aldosterone blockade in chronic kidney disease: can it improve outcome? Curr Opin Nephrol Hypertens. September 2010; 19 (5): 444-9. doi: 10.1097/MNH.0b013e32833ce6d5
119. Fiebeler A, Muller D N, Shagdarsuren E, Luft F C. Aldosterone, mineralocorticoid receptors, and vascular inflammation. Curr Opin Nephrol Hypertens. March 2007; 16 (2): 134-42. doi: 10.1097/MNH.0b013e32801245bb
120. Ting J, Jiang K, Du S, et al. Longitudinal Blood Pressure Patterns From Mid- to Late Life and Late-Life Hearing Loss in the Atherosclerosis Risk in Communities Study. J Gerontol A Biol Sci Med Sci. Mar. 3 2022; 77 (3): 640-646. doi: 10.1093/gerona/glab153
121. Meneses-Barriviera C L, Bazoni J A, Doi M Y, Marchiori LLM. Probable Association of Hearing Loss, Hypertension and Diabetes Mellitus in the Elderly. Int Arch Otorhinolaryngol. October 2018; 22 (4): 337-341. doi: 10.1055/s-0037-1606644
122. Miyata J, Umesawa M, Yoshioka T, Iso H. Association between high systolic blood pressure and objective hearing impairment among Japanese adults: a facility-based retrospective cohort study. Hypertens Res. January 2022; 45 (1): 155-161. doi: 10.1038/s41440-021-00737-8

### Example II

Rescue Treatment with Losartan is Protective Against Noise-Induced Hearing Loss/Losartan Rescues Hearing after Noise

### Overview

### Objective

To investigate the effect of losartan rescue dosing on auditory function after acoustic trauma in a mouse model.

### Study Design

Animal model blinded study.

### Setting

Animal facility, tertiary research center

### Methods

CBA/J mice (age 9-10 weeks, n=8-9 per group) were randomly assigned to mixed male and female groups receiving 1) standard chow, 2) losartan-infused chow (20 mg/kg/day) starting two days prior to noise exposure, or 3) losartan-infused chow (20m/kg/day) starting one hour after completion of noise exposure of two hours of 8-16k octave band noise at 105 dB for males and 107.5 dB for females. The treatment group received losartan infused chow until two weeks post-noise exposure and then transitioned back to normal food. All mice underwent auditory brainstem responses recordings (ABRs) and distortion product otoacoustic emissions (DPOAEs) one day prior to noise exposure and repeated one, seven, fourteen, and thirty days after noise exposure.

### Results

Losartan rescue treatment significantly protected against ABR threshold shifts most robustly at 14 days post-noise exposure at 8 kHz (38.75 dB vs 70.62 dB SPL, p<0.01), 16 kHz (32.5 vs 63.7 dB SPL, p<0.01), and 32 kHz (36.87 dB vs 68.12 dB SPL, p<0.001). These protective effects persisted to 30 days after noise exposure and yielded ABR thresholds that were not significantly different than those in non-noise exposed age matched controls.

### Conclusion

Treatment with losartan is protective against acoustic trauma, whether given before or after noise exposure, in preventing permanent ABR and DPOAE threshold shifts, effects that persist even after cessation of losartan treatment.

### INTRODUCTION

Loss of hearing secondary to environmental factors such as noise remains a major public health concern with audiometric evidence of noise-induced hearing loss (NIHL) in ~25% of adults with measurable hearing loss and in 20% of adults without clinically significant hearing loss (1,2). The pathophysiology of NIHL is multifactorial, involving changes at the level of the cochlear synapses, hair cells, and spiral ganglion neurons (3-7) along with strial microvascular changes (8,9), excitotoxicity (10-13), and inflammation (14-16). In theory, therapies for NIHL that counter multiple such processes have greater potential for efficacy. The renin angiotensin system (RAS) is a pathway conventionally shown to be overactive in diseases states including hypertension, renal disease, pulmonary disease, and neurovascular disease (17-22). Drugs targeting RAS have been shown to decrease inflammation, fibrosis, cerebrovascular ischemia, and oxidative stress in multiple organ systems (17, 23-29) including the inner ear (30-34).

It was recently observed that angiotensin receptor blockade via administration of losartan prior to acoustic trauma significantly protected against auditory brainstem response (ABR) shifts in a murine model (35, 36). However, because loud noise exposure can occur unpredictably, therapies with high translational potential of alleviating NIHL must exert otoprotection after initial acoustic insult. Such rescue dosing strategies are commonly applied in the setting of sudden sensorineural hearing loss with the administration of corticosteroids (37,38).

At this time, there is no approved or effective treatment in the prevention or treatment of noise induced hearing loss. Furthermore, expression of components of the RAS within the cochlea has been minimally studied. Given previous studies and current understanding of mechanisms of noise induced hearing loss, it hypothesized that losartan rescue dosing following acoustic trauma protects against permanent ABR threshold shifts after moderate noise exposure.

### Materials and Methods

### Ethical Approvals

All animal experimental protocols in this study were approved by the University of Iowa Institutional Animal Care and Use Committee (Protocol 3022519).

### Animals and Experimental Cohorts

CBA/J mice are comparatively resistant to age-related hearing loss and a commonly used strain for the study of NIHL (39). Mixed sex, age-matched mice were assigned to three groups: 1) standard chow 2) losartan-infused chow (20 mg/kg/day, equivalent to ~100 mg daily dose in humans) starting two days prior to noise exposure, or losartan-infused chow (20 mg/kg/day) beginning 1 hour after exposure to two hours of 8-16 kHz octave band noise at sound pressure levels (SPL) of 105 dB for males and 107.5 dB for females for the PTS cohort (n=8-9 per group).

Females were subjected to slightly higher dB SPL noise exposure due to well-known effects of estradiol on hearing protection (40). In both cohorts, mice assigned to the treatment group received losartan-infused chow up to 14 days after noise-exposure and were then transitioned to standard chow. ABR and DPOAE testing were done 1 day prior to noise exposure and again at 1, 7, 14, and 30 days after noise exposure. Three mice were excluded from the final analysis: one for which baseline ABR thresholds were found to be severely elevated prior to noise exposure and two that did not experience significant threshold shifts after noise exposure.

### Auditory Brainstem Response 136 (ABR) and Distortion Product Otoacoustic Emission (DPOAE) Recordings

Mice were anesthetized throughout ABR and DPOAE testing by intraperitoneal injection of ketamine/xylazine (75/10 mg/kg) and placed on a heating pad. Recordings were made using the Tucker-Davis Technologies RZ6 Z-Series Bioacoustic System with stimuli from 90 down to 20 dB SPL or until no waveform was present in response to tone bursts at 8-, 16-, and 32-kHz frequencies. ABR thresholds were defined as the lowest sound pressure level required to observe wave 1. DPOAEs were also measured in a subset of animals from each group as previously described (41).

### Results

### Protection Against Noise-Induced Hearing Loss (NIHL) by Losartan Rescue Dosing in Mice

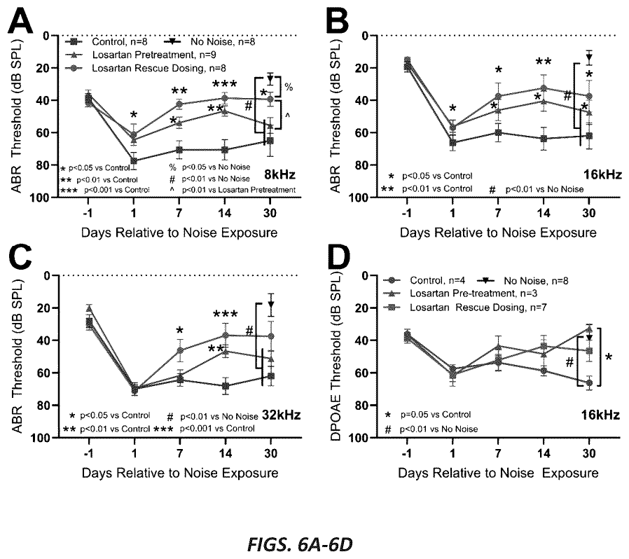

Two hours of moderate acoustic trauma resulted in permanent ABR threshold shifts (PTS) that persisted to 30 days (FIG. 6). In recapitulation of previous findings (35,36), pretreatment with losartan (FIG. 6, green up triangles) significantly blunted ABR shifts recorded at 1, 7, 14, and 30 days after noise exposure compared to control group (blue squares), with the most significant protection seen in this group at 14 days in response to stimuli at 8 kHz (46.66 dB vs 70.62 dB SPL, p<0.01) (FIG. 1A), 16 kHz (40.55 dB vs 63.7 dB SPL, p<0.05) (FIG. 6B), and 32 kHz (46.66 dB vs 68.12 dB SPL, p<0.01) (FIG. 6C). DPOAE thresholds at 30 days after noise exposure were also found to be significantly lower compared to control chow treated mice (FIG. 6D). Though some protection did persist to 30 days, hearing thresholds among mice receiving losartan pretreatment remained significantly impaired at 30 days as compared to non-noise exposed age matched controls (black down triangles) (FIG. 6A-C). In the cohort with losartan treatment starting 1 hour after noise exposure in a rescue strategy (red circles), there was similarly highly significant protection against ABR thresholds shifts at 7, 14, and 30 days, with the highest levels of protection seen at 14 days in response to stimuli at 8 kHz (38.75 dB vs 70.62 dB SPL, p<0.01) (FIG. 6A), 16 kHz (32.5 vs 63.7 dB SPL, p<0.01) (FIG. 6B), and 32 kHz (36.87 dB vs 68.12 db SPL, p<0.001) (FIG. 6C), effects that persisted to 30 days.

In these rescue-treated mice, there was no significant difference in 30-day ABR thresholds at 16 kHz and 32 kHz stimuli compared to thresholds from age-matched non-noise exposed controls (black down triangles) (FIG. 6B-C), suggesting an especially robust protective response.

Given the known sex specific differences in hearing (40), further subgroup analysis of ABRs were completed. Losartan rescue treatment was shown to exert protective effects against any one frequency among both sexes, specifically at 8 kHz (35 dB vs 82.5 dB SPL, p=0.014) in males and at 32 kHz (32.5 dB vs 67.5 dB SPL, p=0.021) in females (Table 6).

TABLE 6
Sub-group Analysis of median ABR thresholds at 14 days after noise exposure by sex
Median ABR thresholds at 14 days after noise exposure compared by sex

| | | | | Małe | | | | | Female | |
|---|---|---|---|---|---|---|---|---|---|---|
| | | Male | | (n = 4) | | | Female | | (n = 5) | |
| | Male | (n = 5) | P-value | Losartan | P-value | Female | (n = 5) | P-value | Losartan | P-value |
| | (n = 4) | Losartan | vs | Rescue | vs | (n = 4) | Losartan | vs | Rescue | vs |
| Frequency | Control | Pretreatment | control | Treatment | control | Control | Pretreatment | control | Treatment | control |
| 8 KHz | 82.5 | 45 | 0.008 | 35 | 0.014 | 57.5 | 55 | 0.28 | 45 | 0.071 |
| (dB HL) | | | | | | | | | | |
| 16 8Hz | 72.5 | 35 | 0.05 | 32.5 | 0.057 | 70 | 45 | 0.095 | 30 | 0.057 |
| (dB HL) | | | | | | | | | | |
| 32 KHz | 70 | 45 | 0.02 | 45 | 0.114 | 67.5 | 55 | 0.25 | 32.5 | 0.021 |
| (dB HL) | | | | | | | | | | |

### Discussion

As a clinical entity that lacks approved pharmacologic therapies, noise-induced hearing loss (NIHL) represents an area of significant clinical potential. As basic studies continue to elucidate cellular and molecular mechanisms through which acoustic trauma leads to hearing loss, drug treatments targeting these mechanisms are becoming increasingly plausible. Losartan is an angiotensin 2 type 1 receptor (AT1R) blocker, that is widely used as a first-line medication for patients with hypertension, chronic kidney disease, and heart failure (42-45). In this preclinical study, a standard dose of losartan (equivalent to ~100 mg daily in humans) was tested as a rescue treatment against NIHL. In addition to recapitulating previous data demonstrating the protective effect of losartan pre-treatment against acoustic trauma, it was found that losartan rescue treatment yielded a robust protection with at least as much efficacy against ABR threshold shifts after noise exposure (FIG. 6). Losartan-induced hearing protection was maintained in the rescue treatment cohort as seen from the persistently significant otoprotection at 30 days that is seen only to a lesser extent in the pre-treatment cohort. The possibility that losartan rescue treatment results in an even more pronounced protection than losartan pre-treatment is supported by the observation that ABR thresholds in these mice at 30 days, but not mice pre-treated with losartan, were not significantly different from ABR thresholds of age matched non-noise exposed mice. Further, despite cessation of losartan treatment at 14 days, otoprotection persisted to at least 30 days, suggesting that blockade of AT1Rs during the acute and subacute period following NIHL is sufficient for durable hearing protection without need for continuing long-term treatment.

The distribution of the receptors and enzymes of the RAS within the cochlea, such as the angiotensin 2 type 1 receptor (AT1R), angiotensin 2 type 2 receptor (AT2R), Mas receptor (MasR), angiotensin converting enzyme (ACE), and angiotensin converting enzyme 2 (ACE2) has not been thoroughly described, although it is likely that at least some of these molecules are expressed in the cochlea including ACE2 on hair cells (46). The classical understanding of the RAS as a primarily renal- and pulmonary-based endocrine system that functions to preserve blood volume and electrolyte homeostasis has been expanded in recent decades to include the understanding of a variety of organ specific effects of RAS signaling in both an endocrine and a paracrine fashion. In this context, the RAS has been studied in various tissues and pathologic states for its deleterious or protective effects as mediated by at least two complementary axes: 1) the conventional ACE/Ang2/AT1R axis that drives vasoconstrictive, inflammatory, and fibrotic effects in response to chronic activation; and 2) the counterregulatory ACE2/Ang-(1-7)/MasR axis that promotes vasodilatory, anti-inflammatory, and anti-fibrotic effects. This protective or counter-regulatory axis also includes signaling through the AT2R, a transmembrane G-protein coupled receptor that also binds Ang II and has been found to counteract signaling of the AT1R, including by dimerization with AT1Rs (47). Interestingly, AT1R has not been shown to be expressed on a transcriptomic level in the hair cells but may be expressed in the pillar and Deiters cells (48). However, it has been shown to be expressed in very small amounts in both hair and supporting cells on single cell RNA sequencing (scRNA seq) (49). Furthermore, expression of AT1R has been more readily, although still minimally, observed after noise exposure (50). This brings into question the possibility of AT1R upregulation in the face of noise-induced ototoxicity.

Alternatively, AT2Rs may be the local target responsible for attenuation of acoustic trauma in the setting of losartan administration. The affinity of angiotensin II is similar for both AT2R and AT1R, but the general distribution of AT2R is much lower than that of AT1R, with drastic decrease in expression after birth, and is primarily seen in neural and renal tissue (51, 52), thus raising the possibility for higher expression levels in the cochlea. AT2Rs have not been found to be expressed on a transcriptomic level in any of the hair cells or supporting cells (48). However, its expression has been detected in hair cells after noise exposure (50) and has been detected in scRNA seq (49). Signaling through the AT2R counteracts that of AT1R signaling. AT2R activation stimulates the activation of phosphotyrosine phosphatases and mitogen-activated protein kinase phosphatase-1 (MKP-1) which in turn inhibits the activity of NF-κB and mitogen-activated protein kinase (MAPK), resulting in its anti-fibrotic, anti-proliferative, and anti-inflammatory properties (52). More notably, AT2R activates a bradykinin-dependent stimulation of nitrous oxide production with a concomitant increase in production of superoxide dismutase, thus resulting in its vasodilatory and antioxidant effects (52).

ACE is an enzyme that converts angiotensin into angiotensin II, with likely expression within the cochlea (46, 48, 49) and shown to be expressed after noise (50). Therefore, it is possible that noise exposure can upregulate the expression of ACE and subsequently increase angiotensin II action, thus contributing to the conventional fibrotic, inflammatory, and ischemic sequelae attributed to NIHL. The MasR is another receptor involved within the RAS system that preferentially and specifically binds to the heptapeptide Ang (1-7), which is produced from angiotensin II by ACE2 and less commonly from angiotensin I through an intermediary via both ACE and ACE2. The MasR has been shown to preferentially be expressed within the brain, testes, and endothelial cells (53) and has been shown to be expressed at a transcriptomic level within the hair and supporting cells 48. Activation of the MasR has been heavily studied due to its activation of the protective arm of the RAS pathway. In the kidney, Ang (1-7) has been shown to decrease phosphorylation and activation of MAPKs, similarly to the action of AT2R activation (54), decrease TGF beta expression (55), and decrease renal oxidative stress (56). Similar effects have been found in the heart (57, 58), lungs (59), and liver (60). In fact, ACE2 61 and MasR knockout (62) reinforced effects seen in the fibrotic and inflammatory arm of the RAS pathway, exemplifying the protective effect that Ang (1-7) and MasR activation provide. Several studies have also shown that ARBs such as telmisartan and olmesartan exert their effects through modulation of the ACE2/Ang (1-7) arm of the RAS pathway to provide both anti-fibrotic and anti-inflammatory effects (63, 64). If inflammation secondary to noise overexposure activates the RAS pathway as in other organ systems (65, 66), losartan administration can be hypothesized to impart several effects. First, losartan administration would decrease binding of Ang II to AT1R and instead shunt towards binding to AT2R which would activate the anti-inflammatory, vasoprotective, and anti-fibrotic arm of the RAS pathway, and in turn decrease the drivers of continued apoptosis and vascular remodeling even after initial acoustic insult. Second, by disruption of a homeostatic negative feedback loop within the RAS, losartan administration can increase upstream metabolites such as Ang I and ACE, thus increasing Ang II concentrations, which would further activate the AT2R and create more substrate for potential generation of Ang-(1-7) via ACE and ACE2. In turn, Ang-(1-7) would bind and activate the MasR, further potentiating the protective axis of the RAS pathway. Third, losartan administration would decrease downstream effectors of the RAS system, namely aldosterone. Aldosterone can serve as an effector of RAS pathway activation and driver of conventional RAS associated pathology in multiple organ systems including the kidneys and cardiovascular system secondary to vascular and tissue remodeling (66-69). Mineralocorticoid receptors have been found to be expressed within the hair cells and supporting cells (48, 49), implying that there may be an aldosterone-mediated effector mechanism at play within the cochlea. Therefore, any action upon the intracochlear mineralocorticoid receptors is likely secondary to systemic signaling cascades rather than local, thus supporting the proposed interplay of systemic RAS signaling with local cochlear signaling.

### BIBLIOGRAPHY

1. Cunningham LL, Tucci DL. Hearing Loss in Adults. N Engl J Med. Dec. 21 2017; 377 (25): 2465-2473. doi: 10.1056/NEJMra1616601
2. Y I C, J E, F S. Vital Signs: Noise-Induced Hearing Loss Among Adults MMWR Morb Mortal Wkly Rep 2017. p. 139-144.
3. Kujawa S G, Liberman M C. Acceleration of age-related hearing loss by early noise exposure: evidence of a misspent youth. J Neurosci. Feb. 15 2006; 26 (7): 2115-23. doi: 10.1523/jneurosci.4985-05.2006
4. Kujawa S G, Liberman M C. Adding insult to injury: cochlear nerve degeneration after “temporary” noise-induced hearing loss. J Neurosci. Nov. 11 2009; 29 (45): 14077-85. doi: 10.1523/jneurosci.2845-09.2009
5. Liberman M C, Kujawa S G. Cochlear synaptopathy in acquired sensorineural hearing loss: Manifestations and mechanisms. Hear Res. June 2017; 349:138-147. doi: 10.1016/j.heares.2017.01.003
6. Fernandez K A, Guo D, Micucci S, De Gruttola V, Liberman M C, Kujawa S G. Noise335 induced Cochlear Synaptopathy with and Without Sensory Cell Loss. Neuroscience. Feb. 10 2020; 427:43-57. doi: 10.1016/j.neuroscience.2019.11.051
7. Kujawa S G, Liberman M C. Synaptopathy in the noise-exposed and aging cochlea: Primary neural degeneration in acquired sensorineural hearing loss. Hear Res. December 2015; 330 (Pt B): 191-9. doi: 10.1016/j.heares.2015.02.009
8. Shin S A, Lyu A R, Jeong S H, Kim T H, Park M J, Park Y H. Acoustic Trauma Modulates 341 Cochlear Blood Flow and Vasoactive Factors in a Rodent Model of Noise-Induced Hearing Loss. Int J Mol Sci. Oct. 25 2019; 20 (21) doi: 10.3390/ijms20215316
9. Shi X, Nuttall A L. Up 343 regulated iNOS and oxidative damage to the cochlear stria vascularis due to noise stress. Brain Res. Mar. 28 2003; 967 (1-2): 1-10. doi: 10.1016/s0006-8993 (02) 04090-8
10. Ma K, Zhang A, She X, et al. Disruption of Glutamate Release and Uptake-Related Protein Expression After Noise-Induced Synaptopathy in the Cochlea. Front Cell Dev Biol. 2021; 9:720902. doi: 10.3389/fcell.2021.720902
11. Hu N, Rutherford M A, Green S H. Protection of cochlear synapses from noise-induced excitotoxic trauma by blockade of Ca (2+)-permeable AMPA receptors. Proc Natl Acad Sci USA. Feb. 18 2020; 117 (7): 3828-3838. doi: 10.1073/pnas. 1914247117
12. Hakuba N, Koga K, Gyo K, Usami S I, Tanaka K. Exacerbation of noise-induced hearing loss in mice lacking the glutamate transporter GLAST. J Neurosci. Dec. 1 2000; 20 (23): 8750-3. doi: 10.1523/jneurosci.20-23-08750.2000
13. Kim K X, Payne S, Yang-Hood A, et al. Vesicular Glutamatergic Transmission in Noise-Induced Loss and Repair of Cochlear Ribbon Synapses. J Neurosci. Jun. 5 2019; 39 (23): 4434-4447. doi: 10.1523/jneurosci.2228-18.2019
14. Paciello F, Pisani A, Rolesi R, et al. Noise-Induced Cochlear Damage Involves PPAR Down-Regulation through the Interplay between Oxidative Stress and Inflammation. Antioxidants (Basel). Jul. 26 2021; 10 (8) doi: 10.3390/antiox10081188
15. Fujioka M, Kanzaki S, Okano H J, Masuda M, Ogawa K, Okano H. Proinflammatory cytokines expression in noise-induced damaged cochlea. J Neurosci Res. March 2006; 83 (4): 575-83. doi: 10.1002/jnr.20764
16. Frye M D, Ryan A F, Kurabi A. Inflammation associated with noise-induced hearing loss. J Acoust Soc Am. November 2019; 146 (5): 4020. doi: 10.1121/1.5132545
17. Mezzano S A, Ruiz-Ortega 366 M, Egido J. Angiotensin II and renal fibrosis. Hypertension. September 2001; 38 (3 Pt 2): 635-8. doi: 10.1161/hy09t1.094234
18. Gupta D, Kumar A, Mandloi A, Shenoy V. Renin angiotensin aldosterone system in pulmonary fibrosis: Pathogenesis to therapeutic possibilities. Pharmacol Res. December 2021; 174:105924. doi: 10.1016/j.phrs.2021.105924
19. Wang J, Chen L, Chen B, et al. Chronic Activation of the Renin-Angiotensin System Induces Lung Fibrosis. Sci Rep. Oct. 23 2015; 5:15561. doi: 10.1038/srep15561
20. Abiodun O A, Ola M S. Role of brain renin angiotensin system in neurodegeneration: An update. Saudi J Biol Sci. March 2020; 27 (3): 905-912. doi: 10.1016/j.sjbs.2020.01.026
21. Patel S, Rauf A, Khan H, Abu-Izneid T. Renin-angiotensin-aldosterone (RAAS): The ubiquitous system for homeostasis and pathologies. Biomed Pharmacother. October 2017; 94:317-325. doi: 10.1016/j.biopha.2017.07.091
22. Nakagawa P, Sigmund C D. How Is the Brain Renin-Angiotensin System Regulated? Hypertension. July 2017; 70 (1): 10-18. doi: 10.1161/hypertensionaha.117.08550
23. Díez J, Querejeta R, López B, González A, Larman M, Martínez Ubago J L. Losartan381 dependent regression of myocardial fibrosis is associated with reduction of left ventricular chamber stiffness in hypertensive patients. Circulation. May 28 2002; 105 (21): 2512-7. doi: 10.1161/01.cir.0000017264.66561.3d
24. Miguel-Carrasco J L, Beaumont J, San José G, et al. Mechanisms underlying the cardiac antifibrotic effects of losartan metabolites. Sci Rep. Feb. 3 2017; 7:41865. doi: 10.1038/srep41865
25. Pacurari M, Kafoury R, Tchounwou P B, Ndebele K. The Renin-Angiotensin-aldosterone system in vascular inflammation and remodeling. Int J Inflam. 2014; 2014:689360. doi: 10.1155/2014/689360
26. Bennion D M, Haltigan E, Regenhardt R 389 W, Steckelings U M, Sumners C. Neuroprotective mechanisms of the ACE2-angiotensin-(1-7)-Mas axis in stroke. Curr Hypertens Rep. February 2015; 17 (2): 3. doi: 10.1007/s11906-014-0512-2
27. AlQudah M, Hale T M, Czubryt M P. Targeting the renin-angiotensin-aldosterone system in fibrosis. Matrix Biol. September 2020; 91-92:92-108. doi: 10.1016/j.matbio.2020.04.005
28. Unger T. Inhibiting renin-angiotensin in the brain: the possible therapeutic implications. Blood Press Suppl. 2001; 1:12-6. doi: 10.1080/080370501750066453
29. Loera-Valencia R, Eroli F, Garcia-Ptacek S, Maioli S. Brain Renin-Angiotensin System as Novel and Potential Therapeutic Target for Alzheimer's Disease. Int J Mol Sci. Sep. 20 2021; 22(18) doi: 10.3390/ijms221810139
30. Lee C H, Lee S M, Kim S Y. Telmisartan Attenuates Kanamycin-Induced Ototoxicity in Rats. Int J Mol Sci. Nov. 24 2021; 22 (23) doi: 10.3390/ijms222312716
31. Cortada M, Wei E, Jain N, Levano S, Bodmer D. Telmisartan Protects Auditory Hair Cells from Gentamicin-Induced Toxicity in vitro. Audiol Neurootol. 2020; 25 (6): 297-308. doi: 10.1159/000506796
32. Cosgrove D, Gratton M A, Madison J, et al. Dual inhibition of the endothelin and angiotensin receptor ameliorates renal and inner ear pathologies in Alport mice. J Pathol. July 2023; 260 (3): 353-364. doi: 10.1002/path.6087
33. Meyer zum Gottesberge A M, Massing T, Sasse A, Palma S, Hansen S. Zucker diabetic fatty rats, a model for type 2 diabetes, develop an inner ear dysfunction that can be attenuated by losartan treatment. Cell Tissue Res. November 2015; 362 (2): 307-15. doi: 10.1007/s00441-015-2215-7
34. Wu L, Vasilijic S, Sun Y, e 410 t al. Losartan prevents tumor-induced hearing loss and augments radiation efficacy in NF2 schwannoma rodent models. Sci Transl Med. Jul. 14 2021; 13(602)doi:10.1126/scitranslmed.abd4816
35. Kempton T, Hunger B, Hua R, et al. Physiologic Otoprotection via Renin-Angiotensin system Blockade in Noise-induced Hearing Loss.
36. Chu C, Eckard P, Hua R, et al. Effects of Angiotensin Receptor Blockade on Cochlear Synaptopathy Following Noise Overexposure.
37. Wei B P, Stathopoulos D, O'Leary S. Steroids for idiopathic sudden sensorineural hearing loss. Cochrane Database Syst Rev. Jul. 2 2013; 2013 (7): Cd003998. doi: 10.1002/14651858.CD003998.pub3
38. Kuhn M, Heman-Ackah S E, Shaikh J A, Roehm P C. Sudden sensorineural hearing loss: a review of diagnosis, treatment, and prognosis. Trends Amplif. September 2011; 15 (3): 91-105. doi: 10.1177/1084713811408349
39. Park C R, Willott J F, Walton J P. Age-related changes of auditory sensitivity across the life span of CBA/CaJ mice. Hear Res. January 2024; 441:108921. doi: 10.1016/j.heares.2023.108921
40. Shuster B, Casserly R, Lipford E, et al. Estradiol Protects against Noise-Induced Hearing Loss and Modulates Auditory Physiology in Female Mice. Int J Mol Sci. Nov. 11 2021; 22 (22) doi: 10.3390/ijms222212208
41. Iwasa Y, Klimara M J, Yoshimura H, et al. Mutation-agnostic RNA interference with engineered replacement rescues Tmc1-related hearing loss. Life Sci Alliance. March 2023; 6 (3) doi: 10.26508/1sa.202201592
42. Dahlof B, Devereux R B, Kjeldsen S E, et al. Cardiovascular morbidity and mortality in the Losartan Intervention For Endpoint reduction in hypertension study (LIFE): a randomized trial against atenolol. Lancet. Mar. 23 2002; 359 (9311): 995-1003. doi: 10.1016/s0140-6736 (02) 08089-3
43. Owens P, Kelly L, Nallen R, Ryan D, Fitzgerald D, O'Brien E. Comparison of antihypertensive and metabolic effects of losartan and losartan in combination with hydrochlorothiazide—a randomized controlled trial. J Hypertens. March 2000; 18 (3): 339-45. doi: 10.1097/00004872-200018030-00014
44. Brenner B M, Cooper M E, de Zeeuw D, et al. Effects of losartan on renal and cardiovascular outcomes in patients with type 2 diabetes and nephropathy. N Engl J Med. Sep. 20 2001; 345 (12): 861-9. doi: 10.1056/NEJMoa011161
45. Moen M D, Wagstaff A J. Losartan: a review of its use in stroke risk reduction in patients with hypertension and ventricular left hypertrophy. Drugs. 2005; 65 (18): 2657-74. doi: 10.2165/00003495-200565180-00012
46. Jeong M, Ocwieja K E, Han D, et al. Direct SARS-COV-2 infection of the human inner ear may underlie COVID-19-associated audiovestibular dysfunction. Commun Med (Lond). 2021; 1 (1): 44. doi: 10.1038/s43856-021-00044-w
47. Villela D, Leonhardt J, Patel N, et al. Angiotensin type 2 receptor (AT2R) and receptor Mas: a complex liaison. Clin Sci (Lond). February 2015; 128 (4): 227-34. doi: 10.1042/cs20130515
48. Liu H, Chen L, Giffen K P, et al. Cell-Specific Transcriptome Analysis Shows That Adult Pillar and Deiters' Cells Express Genes Encoding Machinery for Specializations of Cochlear Hair Cells. Front Mol Neurosci. 2018; 11:356. doi: 10.3389/fnmol.2018.00356
49. Kolla L, Kelly M C, Mann Z F, et al. Characterization of the development of the mouse cochlear epithelium at the single cell level. Nat Commun. May 13 2020; 11 (1): 2389. doi: 10.1038/s41467-020-16113-y
50. Cederroth C R, Park J S, Basinou V, 456 et al. Circadian Regulation of Cochlear Sensitivity to Noise by Circulating Glucocorticoids. Curr Biol. Aug. 5 2019; 29 (15): 2477-2487.e6. doi: 10.1016/j.cub.2019.06.057
51. de Kloet A D, Steckelings U M, Sumners C. Protective Angiotensin Type 2 Receptors in the Brain and Hypertension. Curr Hypertens Rep. June 2017; 19 (6): 46. doi: 10.1007/s11906-017-0746-x
52. Matavelli L C, Siragy H M. AT2 receptor activities and pathophysiological implications. J Cardiovasc Pharmacol. March 2015; 65 (3): 226-32. doi: 10.1097/fjc.0000000000000208
53. Xu P, Sriramula S, Lazartigues E. ACE2/ANG-(1-7)/Mas pathway in the brain: the axis of good. Am J Physiol Regul Integr Comp Physiol. April 2011; 300 (4): R804-17. doi: 10.1152/ajpregu.00222.2010
54. Su Z, Zimpelmann J, Burns K D. Angiotensin-(1-7) inhibits angiotensin I I-stimulated phosphorylation of MAP kinases in proximal tubular cells. Kidney Int. June 2006; 69 (12): 2212-8. doi: 10.1038/sj.ki.5001509
55. Moon J Y, Tanimoto M, Gohda T, et al. Attenuating effect of angiotensin-(1-7) on angiotensin II-mediated NAD (P) H oxidase activation in type 2 diabetic nephropathy of KK472 A(y)/Ta mice. Am J Physiol Renal Physiol. June 2011; 300 (6): F1271-82. doi: 10.1152/ajprenal.00065.2010
56. Farooqui Z, Banday A A. Angiotensin 1-7 exerts antioxidant effects, suppresses Mammalian Target of Rapamycin (mTOR) signaling, and inhibits apoptosis in renal proximal tubular cells. Peptides. February 2024; 172:171136. doi: 10.1016/j.peptides.2023.171136
57. Grobe J L, Mecca A P, Lingis M, et al. Prevention of 477 angiotensin II-induced cardiac remodeling by angiotensin-(1-7). Am J Physiol Heart Circ Physiol. February 2007; 292 (2): H736-42. doi: 10.1152/ajpheart.00937.2006
58. Shah A, Oh Y B, Lee S H, Lim J M, Kim S H. Angiotensin-(1-7) attenuates hypertension in exercise-trained renal hypertensive rats. Am J Physiol Heart Circ Physiol. Jun. 1 2012; 302 (11): H2372-80. doi: 10.1152/ajpheart.00846.2011
59. Shenoy V, Ferreira A J, Qi Y, et al. The angiotensin-converting enzyme 2/angiogenesis- (1-7)/Mas axis confers cardiopulmonary protection against lung fibrosis and pulmonary hypertension. Am J Respir Crit Care Med. Oct. 15 2010; 182 (8): 1065-72. doi: 10.1164/rccm.200912-18400C
60. Pereira R M, Dos Santos R A, Teixeira M M, et al. The renin-angiotensin system in a rat model of hepatic fibrosis: evidence for a protective role of Angiotensin-(1-7). J Hepatol. April 2007; 46 (4): 674-81. doi: 10.1016/j.jhep.2006.10.018
61. Yamamoto K, Ohishi M, Katsuya T, et al. Deletion of angiotensin-converting enzyme 2 accelerates pressure overload-induced cardiac dysfunction by increasing local angiotensin II. Hypertension. April 2006; 47 (4): 718-26. doi: 10.1161/01.HYP.0000205833.89478.5b
62. Gava E, de Castro C H, Ferreira A J, et al. Angiotensin-(1-7) receptor Mas is an essential modulator of extracellular matrix protein expression in the heart. Regul Pept. Apr. 10 2012; 175 (1-3): 30-42. doi: 10.1016/j.regpep.2012.01.001
63. Sukumaran V, Veeraveedu P T, Gurusamy N, et al. Cardioprotective effects of telmisartan against heart failure in rats induced by experimental autoimmune myocarditis through the modulation of angiotensin-converting enzyme-2/angiotensin 1-7/mas receptor axis. Int J Biol Sci. 2011; 7 (8): 1077-92. doi: 10.7150/ijbs.7.1077
64. Sukumaran V, Veeraveedu P 500 T, Gurusamy N, et al. Telmisartan acts through the modulation of ACE-2/ANG 1-7/mas receptor in rats with dilated cardiomyopathy induced by experimental autoimmune myocarditis. Life Sci. Feb. 13 2012; 90 (7-8): 289-300. doi: 10.1016/j.lfs.2011.11.018
65. Benigni A, Cassis P, Remuzzi G. Angiotensin II revisited: new roles in inflammation, immunology and aging. EMBO Mol Med. July 2010; 2 (7): 247-57. doi: 10.1002/emmm.201000080
66. Young M J. Mechanisms of mineralocorticoid receptor-mediated cardiac fibrosis and vascular inflammation. Curr Opin Nephrol Hypertens. March 2008; 17 (2): 174-80. doi: 10.1097/MNH.0b013e3282f56854
67. Verma A, Vaidya A, Subudhi S, Waikar S S. Aldosterone in chronic kidney disease and renal outcomes. Eur Heart J. Oct. 11 2022; 43 (38): 3781-3791. doi: 10.1093/eurheartj/ehac352
68. Toto R D. Aldosterone blockade in chronic kidney disease: can it improve outcome? Curr Opin Nephrol Hypertens. September 2010; 19 (5): 444-9. doi: 10.1097/MNH.0b013e32833ce6d5
69. Fiebeler A, Muller D N, Shagdarsuren E, Luft F C. Aldosterone, mineralocorticoid receptors, and vascular inflammation. Curr Opin Nephrol Hypertens. March 2007; 16 (2): 134-42. doi: 10.1097/MNH.0b013e32801245bb

### Example III

### Angiotensin Receptor Blockade Decreases the Odds for Meniere's and Defining Symptoms—a Multi-National Database Study

### Introduction

Meniere's is in part a microangiopathic hypertensive disease process similar to hypertension-related kidney failure and hypertensive retinopathy. This work describes the protective association of ARBs compared to other first-line anti-hypertensive drugs.

### Objective

Determine if angiotensin receptor blockade (ARB) therapy associates with decreased odds for Meniere's disease and defining symptoms including sensorineural hearing loss (SNHL), tinnitus, and cochlear implantation.

### Study Design

Retrospective cohort database study.

### Setting

TriNetX is a live HIPPA-compliant federated cloud electronic health record research network representing pooled data from 125-million patients from 95 healthcare organizations in the United States, Taiwan, Japan, Brazil, and India.

### Patients

Subjects with Meniere's disease not on an ARB and no other first-line anti-hypertensive. Separate cohorts were generated for patients >30-years-old for each class of first-line anti-hypertensive drugs. Patients were matched using propensity score matching for medical comorbidities and Meniere's risk factors.

### Outcome Measures

Odds-ratios with 95% confidence intervals (OR, 95% CI) for SNHL, tinnitus, cochlear implantation, and Meniere's disease (non-Meniere's cohorts only) after Meniere's diagnosis or starting the anti-hypertensive of interest.

### Results

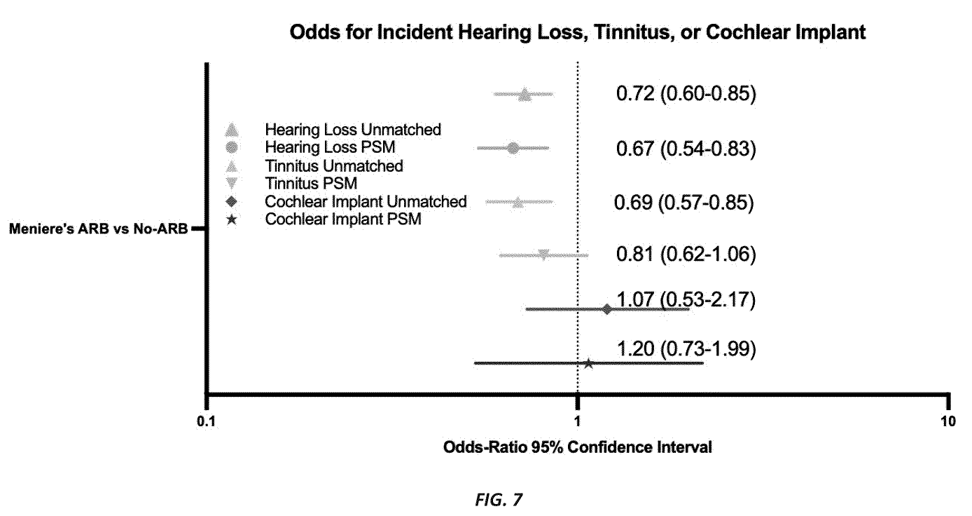

There were 1,958 patients with Meniere's that were on an ARB and no other class of anti-hypertensive that were 1:1 propensity score matched to patients with Meniere's having never taken any anti-hypertensive medication prior to diagnosis. The average age was 67.7 years old, with 56% female patients. The risk for SNHL was 14.20% compared to 17.80% in controls (OR: 0.76, 0.61-0.96). For patients 30-years and older taking an ARB and no other anti-hypertensive medication (n=858,111) versus a thiazide diuretic (n=847,2020) demonstrated a risk for Meniere's of 0.071% versus 0.67% (OR: 0.11, 0.097-0.12). ARBs also associated with protection for SNHL, tinnitus, and cochlear implantation in these cohorts. FIG. 7.

### Conclusions

For Meniere's patients, ARB treatment associates with decreased risk for disease progression. Use of ARBs compared to other first-line anti-hypertensives associates with decreased odds for diagnosis of Meniere's, SNHL, tinnitus, and cochlear implantation.

### Example IV

Interrogate the Functional Role of RAS Components that are Endogenous to the Cochlea in Inducing Protection of Ribbon Synapses Against Excitotoxicity.

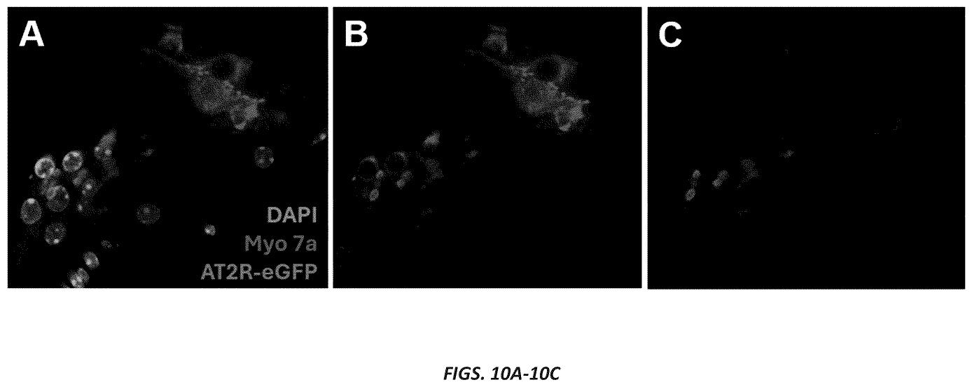

The presence and distribution of RAS elements have yet to be described within the inner ear, due in part to the non-specific nature of available antibodies for these enzymes and receptors. Data utilizing Agt2r reporter mice from Drs. Eric Krause and Annette de Kloet at Georgia State University has revealed the presence Agt2r-GFP+ cells among supporting cells (possibly Dieter cells) along the basal surface of outer hair cells primarily, with some lesser expression observed near inner hair cells (FIGS. 10A-10C). This is consistent with RNAseq data from the online GEAR database revealing greater agtr2 expression in the supporting cells than the hair cells.

### Example V

### Characterize the Role of AT2R Signaling in Losartan's Protective Effects In Vivo Using a Model of NIHL.

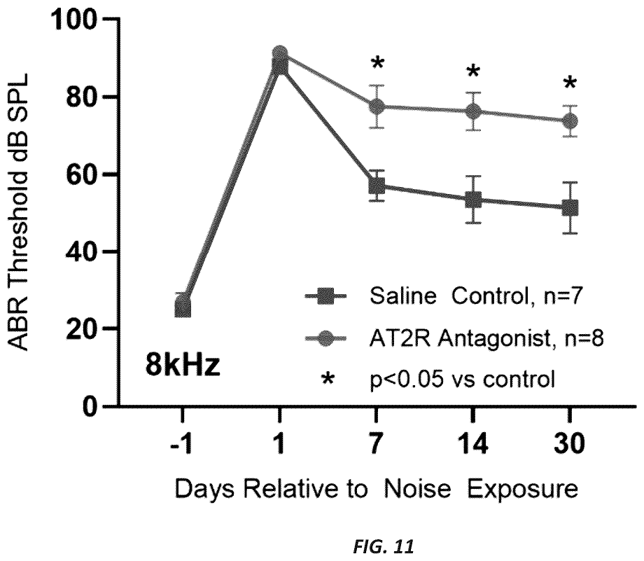

The AT2R has taken on special significance in recent decades for its primary role in counter-regulating the deleterious effects of chronic AT1R activation. Studies of cardiovascular and neurologic pathology employing AT1R blockade for its protective effects have defined a mechanism by which blockade of the type 1 receptor allows for non-competitive binding by Ang II to its type 2 receptor, thus unmasking effects mediated by AT2R-dependent signaling. To this end, data were generated using established noise-induced hearing loss protocol in young adult male and female mice wherein the mice were first implanted with a continuous subcutaneous infusion osmotic minipump to allow for ongoing delivery of sterile saline (control) or a selective AT2R antagonist (PD 123-319, 3 mg/kg/day), followed by noise exposure and measurement of ABR thresholds for up to one month afterward (FIG. 11). Blockade of AT2Rs resulted in significantly worse ABR threshold shifts (p<0.05), indicating a protective role for these receptors.

Unless otherwise defined, all technical and scientific terms used herein have the same meaning as commonly understood by one of ordinary skill in the art to which this invention belongs. Exemplary methods and materials are described herein, although methods and materials similar or equivalent to those described herein can also be used in the practice or testing of the present invention.

Those skilled in the art will recognize, or be able to ascertain, using no more than routine experimentation, numerous equivalents to the specific substances and procedures described herein. Such equivalents are considered to be within the scope of this invention.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

What is claimed is:

1. A method to prevent or treat an auditory disorder comprising administering to a subject in need thereof an angiotensin receptor blocker (ARB), an angiotensin converting enzyme (ACE) inhibitor, an angiotensin II type 2 receptor (AT2R) agonist, a Mas receptor (MasR) agonist or a combination thereof, wherein the ARB is not telmisartan.

2. The method of claim 1, wherein the auditory disorder is hearing loss, tinnitus, Meniere's Disease, endolymphatic hydrops, synaptopathy or an auditory processing disorder.

3. The method of claim 1, wherein the hearing loss is due to acoustic/noise trauma or ototoxicity.

4. The method of claim 1, wherein the hearing loss is sensorineural hearing loss.

5. The method of claim 1, wherein the ARB is Azilsartan, Losartan, Valsartan, Candesartan, Telmisartan, Irbesartan, Olmesartan, Fimasartan, Saprisartan, Eprosartan or a combination thereof.

6. The method of claim 1, wherein the AT2R agonist is buloxibutid, Compound 21, M024, CGP42112, PD-123-319, β-Pro7Ang III, angiotensin-(1-7), alamandine, and [Sar1,Ile8] Angiotensin II or a combination thereof.

7. The method of claim 1, wherein the MasR agonist is Angiotensin-(1-7), [D-Ala7]-Angiotensin-(1-7), p-Amino-phenylalanine-containing analogues of Angiotensin-(1-7), HPβCD-Angiotensin-(1-7), cyclodextran-bound angiotensin-(1-7), BIO101, CGEN-856S, CGEN-857, AVE0779, and CGP42112 or a combination thereof.

8. The method of claim 1, wherein the subject has hypertension.

9. The method of claim 1, wherein the subject does not have hypertension.

10. The method of claim 1, wherein the administration is carried out hours, days or weeks prior to or after the onset of an auditory disorder.

11. The method of claim 1, wherein the administration is carried out hours, days, weeks, months or years after the onset of an auditory disorder.

12. A method to prevent or treat tinnitus, Meniere's Disease or dizziness/vertigo comprising administering to a subject in need thereof an angiotensin receptor blocker (ARB), a calcium channel blocker (CCB), an angiotensin converting enzyme (ACE) inhibitor, an angiotensin II type 2 receptor (AT2R) agonist, a Mas receptor (MasR) agonist or a combination thereof.

13. The method of claim 12, wherein the ARB is Azilsartan, Losartan, Valsartan, Candesartan, Telmisartan, Irbesartan, Olmesartan, Fimasartan, Saprisartan, Eprosartan or a combination thereof.

14. The method of claim 12, wherein the AT2R agonist is buloxibutid, Compound 21, M024, CGP42112, PD-123-319, β-Pro7Ang III, angiotensin-(1-7), alamandine, and [Sar1,Ile8] Angiotensin II or a combination thereof.

15. The method of claim 12, wherein the MasR agonist is Angiotensin-(1-7), [D-Ala7]-Angiotensin-(1-7), p-Amino-phenylalanine-containing analogues of Angiotensin-(1-7), HPβCD-Angiotensin-(1-7), cyclodextran-bound angiotensin-(1-7), BIO101, CGEN-856S, CGEN-857, AVE0779, and CGP42112 or a combination thereof.

16. The method of claim 12, wherein the subject has hypertension.

17. The method of claim 12, wherein the subject does not have hypertension.

18. The method of claim 12, wherein the administration is carried out hours, days or weeks prior to or after the onset of an auditory disorder.

19. The method of claim 12, wherein the administration is carried out hours, days, weeks, months or years after the onset of an auditory disorder.

20. A method to prevent or treat a shift in auditory brainstem response (ABR) from noise induced trauma comprising administering to a subject in need thereof an angiotensin receptor blocker (ARB), an angiotensin converting enzyme inhibitors (ACEi), an angiotensin II type 2 receptor (AT2R) agonist, a Mas receptor agonist or a combination thereof.

21. The method of claim 20, wherein the subject has hypertension.

22. The method of claim 20, wherein the subject does not have hypertension.

23. The method of claim 20, wherein the administration is carried out hours, days or weeks prior to or after the onset of an auditory disorder.

24. The method of claim 20, wherein the administration is carried out hours, days, weeks, months or years after the onset of an auditory disorder.
